(19) United States
(12) Patent Application Publication
(10) Pub. No.: US 2014/0356325 A1
(43) Pub. Date: Dec. 4, 2014

(54) NOVEL 2'-C-METHYL NUCLEOSIDE DERIVATIVE COMPOUNDS

(71) Applicant: Ligand Pharmaceuticals Incorporated, San Diego, CA (US)

(72) Inventors: Lin Zhi, San Diego, CA (US); K. Raja Reddy, San Diego, CA (US); Mark David Erion, Mountainside, NJ (US); Scott Hecker, Del Mar, CA (US)

(73) Assignee: Ligand Pharmaceuticals Incorporated, San Diego, CA (US)

(21) Appl. No.: 14/371,397

(22) PCT Filed: Jan. 8, 2013
(86) PCT No.: PCT/US2013/020696

§ 371 Date: Jul. 9, 2014

Related U.S. Application Data
(60) Provisional application No. 61/586,046, filed on Jan. 12, 2012.

(51) Int. Cl. C07H 19/20; A61K 45/06; A61K 31/7072; C07H 19/10; A61K 31/708

(52) U.S. Cl. CPC C07H 19/20; C07H 19/10; A61K 31/708; A61K 31/7072; A61K 45/06
USPC 424/85.6; 536/26.12; 536/26.14; 514/48; 514/51; 514/4.3; 424/85.7; 514/46

(57) ABSTRACT

Compositions and methods relating to 2′-C-methyl nucleoside 5′-monophosphate derivative compounds are provided. In some embodiments, the novel compounds are useful to treat viral infections. In particular, 2′-C-methyl nucleoside 5′-monophosphate derivative compounds, stereoisomers, and pharmaceutically acceptable salts or prodrugs thereof, their preparation, and their uses for the treatment of viral infection are described.

### FIELD OF THE INVENTION

Compositions and methods in the field of medicine and chemistry are disclosed. Some of the disclosed embodiments are directed towards novel 2′-C-methyl nucleoside 5′-monophosphate derivative compounds, their preparation and their uses. In some embodiments, the novel compounds are useful to treat viral infections.

### BACKGROUND

Hepatitis C is a viral disease that causes inflammation of the liver that may lead to cirrhosis, primary liver cancer and other long-term complications. Nucleosides are a well-recognized class of compounds shown to be effective against a variety of viral infections, including hepatitis B, HIV, and herpes. Several nucleosides are reported to inhibit hepatitis C (HCV) virus replication, including ribavirin, which currently is marketed as a drug combination with various interferons, and nucleosides containing a 2′-C-methyl ribose sugar.

Nucleosides are generally effective as antiviral agents following conversion of the nucleoside to the corresponding nucleoside 5′-triphosphate (NTP). Conversion occurs inside cells through the action of various intracellular kinases. The first step, i.e. conversion of the nucleoside to the 5′-monophosphate (NMP) is generally the slow step and involves a nucleoside kinase, which is encoded by either the virus or host. Conversion of the NMP to the NTP is generally catalyzed by host nucleotide kinases. The NTP interferes with viral replication through inhibition of viral polymerases and/or via incorporation into a growing strand of DNA or RNA followed by chain termination.

Use of nucleosides to treat viral liver infections is often complicated by one of two problems. In some cases, the desired nucleoside is a good kinase substrate and accordingly produces NTP in the liver as well as other cells and tissues throughout the body. Since NTP production is often associated with toxicity, efficacy can be limited by extrahepatic toxicities. In other cases, the desired nucleoside is a poor kinase substrate so is not efficiently converted into the NMP and ultimately into the NTP.

For instance, U.S. Pat. No. 6,312,662 and U.S. Pat. No. 7,666,855 disclose the use of certain phosphate prodrugs for the liver-specific delivery of various drugs including nucleosides for the treatment of patients with liver diseases such as hepatitis C, hepatitis B and hepatocellular carcinoma.

### SUMMARY OF THE INVENTION

Novel 2′-C-methyl nucleoside 5′-monophosphate derivatives, their preparation and their uses for the treatment of hepatitis C viral infections are described. Some embodiments include use of the compound to treat certain viral infections.

Some embodiments include a compound of Formula I or II:

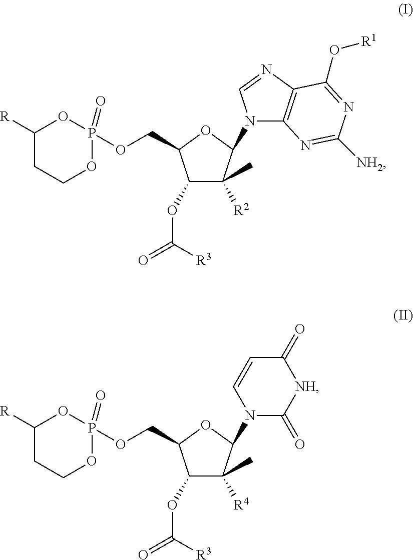

wherein: R is an optionally substituted phenyl or an optionally substituted pyridyl;

R¹ is selected from the group consisting of hydrogen, a C₁-C₁₀ alkyl, C₁-C₁₀ alkenyl, and a C₁-C₁₀ heteroalkyl;

R² is selected from the group consisting of OR⁵, acyloxy, and F;

R³ is a C₁-C₆ alkyl or a C₁-C₆ heteroalkyl, or R² and R³ can be linked to form a —O— bond;

R⁴ is selected from the group consisting of OR⁵ and acyloxy; or R⁴ and R³ can be linked to form a —O— bond;

R⁵ is selected from the group consisting of hydrogen, a C₁-C₆ alkyl, a C₁-C₆ haloalkyl, and a C₁-C₆ heteroalkyl; the stereoisomers and the pharmaceutically acceptable salts thereof.

In some embodiments, wherein R¹ is hydrogen.

In some embodiments, wherein R¹ is methyl.

In some embodiments, wherein R¹ is ethyl.

In some embodiments, R² and R³ are linked to form a —O— bond.

In some embodiments, R⁵ is hydrogen.

In some embodiments, R² or R⁴ is fluorine.

In some embodiments, R² or R⁴ is methyl.

In some embodiments, R² or R⁴ is methyl.

In some embodiments, wherein R³ is methyl.

In some embodiments, wherein R³ is isopropyl.

In some embodiments, wherein R³ is ethyl.

In some embodiments, wherein R³ is cyclopentyl.

Some embodiments include a pharmaceutical composition comprising a therapeutically effective amount of a compound provided herein, and at least one pharmaceutically acceptable carrier, diluent, or lubricant.

Some embodiments include a method of treating a viral infection comprising administering to a subject suffering from the viral infection a compound provided herein.

Some embodiments include a method of treating a viral infection comprising administering to a subject suffering from the viral infection a pharmaceutical composition as provided herein.

In some embodiments, the viral infection is a viral infection of the liver.

In some embodiments, the viral infection is an RNA-dependent RNA viral infection.

In some embodiments, the viral infection is HCV.

Some embodiments include a method of inhibiting viral replication in a human patient comprising administering to said human patient a therapeutically effective amount of a compound provided herein.

Some embodiments include a method of inhibiting viral replication in a human patient comprising administering to said human patient a therapeutically effective amount of a pharmaceutical composition as provided herein.

In some embodiments, the viral replication is RNA-dependent RNA viral replication.

In some embodiments, the compounds provided herein are used in combination with a with a therapeutically effective amount of a second agent that is active against HCV.

In some embodiments, the second agent that is active against HCV is ribavirin, levovirin, viramidine, thymosin alpha-1, interferon-β, an inhibitor of NS3 serine protease, an inhibitor of inosine monophosphate dehydrogenase, interferon-α, or pegylated interferon-α, alone or in combination with ribavirin or levovirin.

In some embodiments, the second agent that is active against HCV is interferon-α or pegylated interferon-α, alone or in combination with ribavirin or levovirin.

In some embodiments, the second agent is amantadine or a 1′-C, 2′-C-, or 3′-C-branched ribonucleoside.

In some embodiments, the 1′-C, 2′-C-, or 3′-C-branched ribonucleoside is 2′-C-methylcytidine, 2′-C-methyluridine, 2′-C-methyladenosine, 2′-C-methylguanosine, or 9-(2-C-methyl-β-D-ribofuranosyl)-2,6-diaminopurine.

### DETAILED DESCRIPTION

Some embodiments include Formula I and II, stereoisomers, pharmaceutically acceptable salts or prodrugs thereof or pharmaceutically acceptable salts of the prodrugs as represented by Formula I and II:

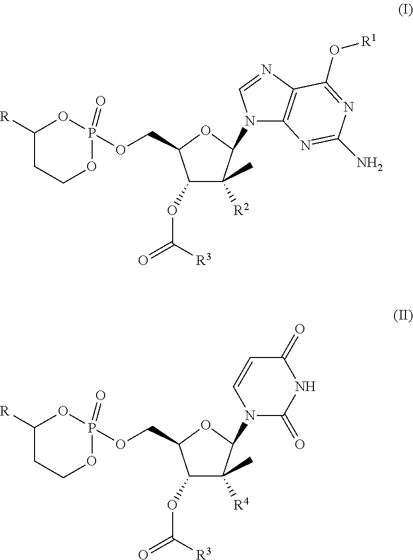

wherein: R is an optionally substituted phenyl or an optionally substituted pyridyl;

R¹ is selected from the group consisting of hydrogen, a C₁-C₁₀ alkyl, C₁-C₁₀ alkenyl, and a C₁-C₁₀ heteroalkyl;

R² is selected from the group consisting of OR⁵, acyloxy, and F;

R³ is a C₁-C₆ alkyl or a C₁-C₆ heteroalkyl, or R² and R³ can be linked to form a —O— bond;

R⁴ is selected from the group consisting of OR⁵ and acyloxy; or R⁴ and R³ can be linked to form a —O— bond;

R⁵ is selected from the group consisting of hydrogen, a C₁-C₆ alkyl, a C₁-C₆ haloalkyl, and a C₁-C₆ heteroalkyl; the stereoisomers and the pharmaceutically acceptable salts thereof.

The compounds described herein are exemplary embodiments but the compounds are not limited to these illustrative compounds. The compounds provided herein are generally prodrugs, as described elsewhere herein. The compounds are shown without depiction of stereochemistry since the compounds are biologically active as the diastereomeric mixture or as a single stereoisomer.

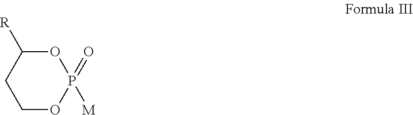

In some embodiments, M may be selected from the group consisting of:

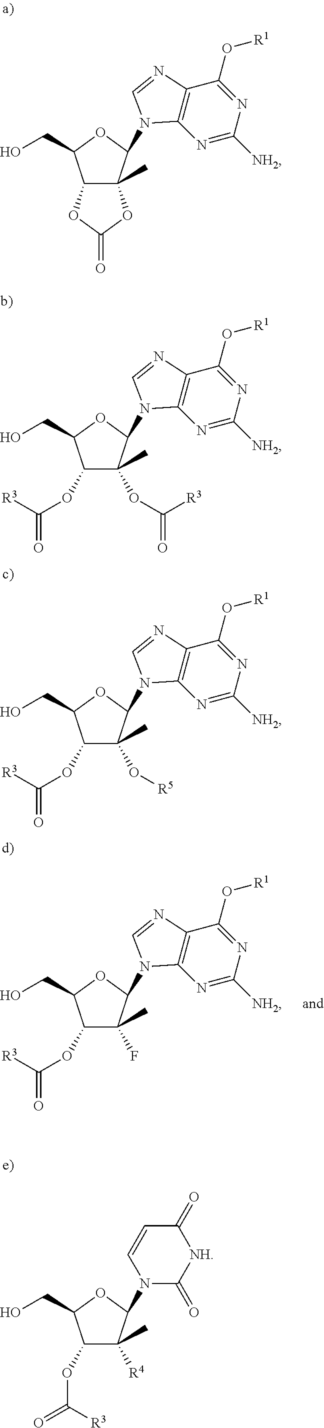

In some embodiments, R is phenyl, 3-chlorophenyl, 3,5-dichlorophenyl, 2,3-dichlorophenyl, 4-pyridyl, 3-pyridyl, or 2-pyridyl.

Moreover, the embodiments of the compounds of Formulas I, II, and III can be used for inhibiting viral replication. In some embodiments, the compounds of Formulas I, II, and III can be used for inhibiting RNA-dependent RNA viral replication. In some embodiments, the compounds of Formulas I, II, and III can be used for inhibiting HCV replication.

In some embodiments, the compounds provided herein can be used for treating viral infections. In a further aspect, compounds provided herein can be used for treating RNA-dependent RNA viral infection. In some embodiments, the compounds provided herein can be used for treating HCV infection.

In some embodiments, the compounds provided herein can be used for treating viral infections of the liver. In some embodiments, the compounds provided herein can be used for treating RNA-dependent RNA viral infection in the liver. In some embodiments, the compounds provided herein can be used for treating HCV infection in the liver.

In some embodiments, inhibition of viral replication is measured in serum. Increased viral titer reduction is associated with decreased generation of viral mutants which are associated with drug resistance.

In some embodiments, the compounds provided herein can be used for preventing the onset of symptoms associated with a viral infection.

In some embodiments, the compounds described herein are prodrugs, activation of which results in the production of a nucleoside monophosphate (NMP). NMPs are frequently further phosphorylated inside the hepatocyte to the biologically active nucleoside triphosphate (NTP). Drug elimination from the hepatocyte typically entails degradation of phosphorylated metabolites back to a species capable of being transported out of the hepatocyte and into the blood for elimination by the kidney or into the bile for biliary excretion. Often with nucleoside-based drug the phosphorylated metabolites are dephosphorylated to the uncharged nucleoside.

Nucleosides that leak back into the systemic circulation result in systemic exposure. If the nucleoside is active systemically, e.g. through entry into virally infected cells and phosphorylation to the active species, escape of the nucleoside from the liver leads to biological activity outside of the liver (i.e. extrahepatic tissues, blood cells). In this case, prodrugs provided herein can be effective for treating diseases outside of the liver, e.g. viral infections. Since many nucleosides exhibit poor oral bioavailability due to breakdown in the gastrointestinal tract either enzymatically (e.g. deamination by adenosine deaminase) or chemically (e.g. acid instability), the prodrug can be used for oral drug delivery. Moreover, given that the prodrugs in some cases are broken down slowly relative to e.g. most ester based prodrugs, the prodrugs could advantageously result in slow, sustained systemic release of the nucleoside.

In other cases, however, systemic exposure to the nucleoside can result in toxicity. This can be minimized by selecting nucleosides that are preferentially excreted through the bile or nucleosides that are unable to undergo phosphorylation in tissues or nucleosides that undergo rapid intrahepatic metabolism to a biologically inactive metabolite. Some enzymes in the hepatocyte are present that can degrade nucleosides and therefore minimize exposure (e.g. Phase I and Phase II enzymes). One example is adenosine deaminase, which can deaminate some adenosine-based nucleosides to produce the corresponding inosine analogue. Rapid intracellular deamination of the nucleoside following its dephosphorylation to the nucleoside limits systemic exposure to the nucleoside and diminishes the risk of toxicity.

Methods described in Examples A-D are used to test activation of compounds provided herein. Methods used in Example E are used to evaluate the ability of compounds provided herein to generate NTPs.

HCV replication in human liver tissue is evaluated in Example F. Liver specificity of the prodrugs relative to the nucleosides was measured by methods in Example G.

Tissue distribution can be determined according to methods in Example H. Oral bioavailability was determined by methods described in Example I. The susceptibility of nucleoside analogs to metabolism can be determined as in Example J.

In some embodiments, the RNA-dependent RNA viral infection is a positive-sense single-stranded RNA-dependent viral infection. In another aspect, the positive-sense single-stranded RNA-dependent RNA viral infection is Flaviviridae viral infection or Picornaviridae viral infection. In a subclass of this class, the Picornaviridae viral infection is rhinovirus infection, poliovirus infection, or hepatitis A virus infection. In a second subclass of this class, the Flaviviridae viral infection is selected from the group consisting of hepatitis C virus infection, yellow fever virus infection, dengue virus infection, West Nile virus infection, Japanese encephalitis virus infection, Banzi virus infection, and bovine viral diarrhea virus infection. In a subclass of this subclass, the Flaviviridae viral infections hepatitis C virus infection.

In some embodiments, the compounds provided herein can be used to enhance the oral bioavailability of the parent drug. In some embodiments, the compounds provided herein can be used to enhance the oral bioavailability of the parent drug by at least 5%. In some embodiments, the compounds provided herein can be used to enhance the oral bioavailability of the parent drug by at least 10%. In another aspect, oral bioavailability is enhanced by 50% compared to the parent drug administered orally. In a further aspect, the oral bioavailability is enhanced by at least 100%.

In some embodiments, the compounds provided herein can be used to increase the therapeutic index of a drug.

In some embodiments, the compounds provided herein can be used to bypass drug resistance.

In some embodiments, the compounds provided herein can be used to treat cancer.

### DEFINITIONS

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, pharmaceutical preparation, formulation, and delivery, and treatment of patients.

The term “alkyl” refers to saturated aliphatic groups including straight-chain, branched chain and cyclic groups, up to and including 10 carbon atoms. Suitable alkyl groups include methyl, ethyl, n-propyl, isopropyl, and cyclopropyl. The alkyl may be optionally substituted with 1-3 substituents.

The term “optionally substituted” or “substituted” includes groups substituted by one to four substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halogen, lower alkylthio, oxo, lower acylalkyl, lower carboxy esters, carboxyl, -carboxamido, nitro, lower acyloxy, lower aminoalkyl, lower alkylaminoaryl, lower alkylaryl, lower alkylaminoalkyl, lower alkoxyaryl, lower acylamino, lower aralkylamino, lower alkylsulfonyl, lower-carboxamidoalkylaryl, lower-carboxamidoaryl, lower hydroxyalkyl, lower haloalkyl, lower alkylaminoalkylcarboxy-, lower aminocarboxamidoalkyl-, cyano, lower alkoxyalkyl, lower perhaloalkyl, and lower arylalkyloxyalkyl. “Substituted aryl” and “substituted heteroaryl” refers to aryl and heteroaryl groups substituted with 1-6 substituents. These substituents are selected from the group consisting of lower alkyl, lower alkoxy, lower perhaloalkyl, halogen, hydroxy, cyano, and amino.

The term “heteroalkyl” refer to alkyl groups containing at least one heteroatom, in a further aspect are 1 to 3 heteroatoms. Suitable heteroatoms include oxygen, sulfur, and nitrogen.

The term “acyloxy” refers to —OC(O)R where R is alkyl, or heteroalkyl.

The term “alkoxy” or “alkyloxy-” refers to —OR where R is alkyl, or heteroalkyl, all optionally substituted.

The term “carboxyl” refers to —C(O)OH.

The term “oxo” refers to ═O in an alkyl or heterocycloalkyl group.

The term “amino” refers to —NRR′ where R and R′ are independently selected from hydrogen, alkyl, aryl, aralkyl and heterocycloalkyl, all except H are optionally substituted; and R and R′ can form a cyclic ring system.

The term “halogen” or “halo” refers to —F, —Cl, —Br and —I.

The term “alkenyl” refers to unsaturated groups which have 2 to 12 atoms and contain at least one carbon-carbon double bond and includes straight-chain, branched-chain and cyclic groups. Alkenyl groups may be optionally substituted. Suitable alkenyl groups include allyl. “1-Alkenyl” refers to alkenyl groups where the double bond is between the first and second carbon atom. If the 1-alkenyl group is attached to another group, e.g. it is a W substituent attached to the cyclic phosphate, it is attached at the first carbon.

The term “alkynyl” refers to unsaturated groups which have 2 to 12 atoms and contain at least one carbon-carbon triple bond and includes straight-chain, branched-chain and cyclic groups. Alkynyl groups may be optionally substituted. Suitable alkynyl groups include ethynyl. “1-Alkynyl” refers to alkynyl groups where the triple bond is between the first and second carbon atom. If the 1-alkynyl group is attached to another group, e.g. it is a W substituent attached to the cyclic phosphate, it is attached at the first carbon.

The term “alkylene” refers to a divalent straight chain, branched chain or cyclic saturated aliphatic group. In one aspect the alkylene group contains up to and including 10 atoms. In another aspect the alkylene chain contains up to and including 6 atoms. In a further aspect the alkylene groups contains up to and including 4 atoms. The alkylene group can be either straight, branched or cyclic. The alkylene may be optionally substituted with 1-3 substituents.

The term “aminoalkyl-” refers to the group NR₂-alk- wherein “alk” is an alkylene group and R is selected from —H, alkyl, aryl, aralkyl, and heterocycloalkyl.

The term “alkylaminoalkyl-” refers to the group alkyl-NR-alk- wherein each “alk” is an independently selected alkylene, and R is H or lower alkyl. “Lower alkylaminoalkyl-” refers to groups where the alkyl and the alkylene group is lower alkyl and alkylene, respectively.

The term “alkoxyalkyl-” or “alkyloxyalkyl-” refer to the group alkyl-O-alk- wherein “alk” is an alkylene group. In “lower alkoxyalkyl-”, each alkyl and alkylene is lower alkyl and alkylene, respectively.

The terms “alkylthio-” refers to the group alkyl-S—.

The term “alkylthioalkyl-” refers to the group alkyl-S-alk- wherein “alk” is an alkylene group. In “lower alkylthioalkyl-” each alkyl and alkylene is lower alkyl and alkylene, respectively.

The term “amido” refers to the NR₂ group next to an acyl or sulfonyl group as in NR₂—C(O)—, RC(O)—NR¹—, NR₂—S(═O)₂— and RS(═O)₂—NR¹—, where R and R¹ include —H, alkyl, aryl, aralkyl, and heterocycloalkyl.

The term “carboxamido” refer to NR₂—C(O)— and RC(O)—NR¹—, where R and R¹ include —H, alkyl, aryl, aralkyl, and heterocycloalkyl. The term does not include urea, —NR—C(O)—NR—.

The term “acylalkyl” refers to an alkyl-C(O)-alk-, where “alk” is alkylene.

The term “perhalo” refers to groups wherein every C—H bond has been replaced with a C-halo bond on an aliphatic or aryl group. Suitable perhaloalkyl groups include —CF₃ and —CFCl₂.

The phrase “therapeutically effective amount” means an amount of a compound or a combination of compounds that ameliorates, attenuates or eliminates one or more of the symptoms of a particular disease or condition or prevents, modifies, or delays the onset of one or more of the symptoms of a particular disease or condition.

The term “pharmaceutically acceptable salt” includes salts of compounds of Formula I and its prodrugs derived from the combination of a compound provided herein and an organic or inorganic acid or base. Suitable acids include acetic acid, adipic acid, benzenesulfonic acid, (+)-7,7-dimethyl-2-oxobicyclo[2.2.1]heptane-1-methanesulfonic acid, citric acid, 1,2-ethanedisulfonic acid, dodecyl sulfonic acid, fumaric acid, glucoheptonic acid, gluconic acid, glucuronic acid, hippuric acid, hydrochloride hemiethanolic acid, HBr, HCl, HI, 2-hydroxyethanesulfonic acid, lactic acid, lactobionic acid, maleic acid, methanesulfonic acid, methylbromide acid, methyl sulfuric acid, 2-naphthalenesulfonic acid, nitric acid, oleic acid, 4,4′-methylenebis[3-hydroxy-2-naphthalenecarboxylic acid], phosphoric acid, polygalacturonic acid, stearic acid, succinic acid, sulfuric acid, sulfosalicylic acid, tannic acid, tartaric acid, terphthalic acid, and p-toluenesulfonic acid.

The term “naturally-occurring L-amino acid” refers to those amino acids routinely found as components of proteinaceous molecules in nature, including alanine, valine, leucine, isoleucine, proline, phenylalanine, tryptophan, methionine, glycine, serine, threonine, cysteine, tyrosine, asparagine, glutamine, aspartic acid, glutamic acid, lysine, arginine and histidine. In one aspect, this term is intended to encompass L-amino acids having only the amine and carboxylic acid as charged functional groups, i.e., alanine, valine, leucine, isoleucine, proline, phenylalanine, tryptophan, methionine, glycine, serine, threonine, cysteine and tyrosine. In another aspect they are alanine, valine, leucine, isoleucine, proline, phenylalanine, and glycine. In a further aspect, it is valine.

The term “patient” refers to an animal being treated including a mammal, such as a dog, a cat, a cow, a horse, a sheep, and a human. Another aspect includes a mammal, both male and female.

The term “prodrug” as used herein refers to any compound that when administered to a biological system generates a biologically active compound as a result of spontaneous chemical reaction(s), enzyme catalyzed chemical reaction(s), and/or metabolic chemical reaction(s), or a combination of each. Standard prodrugs are formed using groups attached to functionality, e.g. HO—, HS—, HOOC—, R₂N—, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other known varieties of prodrugs. Such prodrugs of the compounds of Formula I fall within this scope. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980.

The term “cyclic phosphate ester of 1,3-propanediol”, “cyclic phosphate diester of 1,3-propanediol”, “2 oxo 2λ^{5 [}1,3,2]dioxaphosphorinane”, “2-oxo-[1,3,2]-dioxaphosphorinane”, or “dioxaphosphorinane” refers to the following:

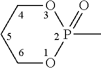

The term “cis” stereochemistry refers to the spatial relationship of the R group and the substituent attached to the phosphorus atom via an exocyclic single bond on the six membered 2-oxo-phosphorinane ring. The structures A and B below show two possible cis-isomers of 2- and 4-substituted 2-oxo-phosphorinane. Structure A shows cis-isomer of (2S,4R)-configuration whereas structure B shows cis-isomer of (2R,4S)-configuration.

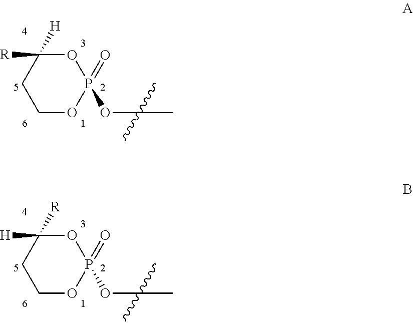

The term “trans” stereochemistry refers to the spatial relationship of the R group and the substituent attached to the phosphorus atom via an exocyclic single bond on the six membered 2-oxo-phosphorinane ring. The structures C and D below show two possible trans-isomers of 2- and 4-substituted 2-oxo-phosphorinane. Structure C shows trans-isomer of (2S,4S)-configuration whereas structure D shows trans-isomer of (2R,4R)-configuration.

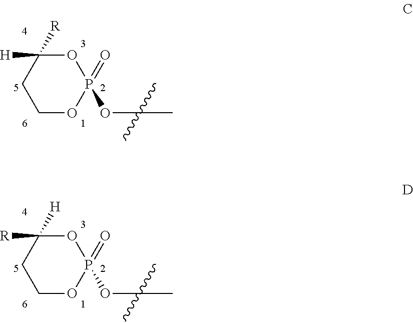

The term “percent enantiomeric excess (% ee)” refers to optical purity. It is obtained by using the following formula:

[R]−[S]×100=% R−% S

[R]+[S]

where [R] is the amount of the R isomer and [S] is the amount of the S isomer. This formula provides the % ee when R is the dominant isomer.

The term “enantioenriched” or “enantiomerically enriched” refers to a sample of a chiral compound that consists of more of one enantiomer than the other. The extent to which a sample is enantiomerically enriched is quantitated by the enantiomeric ratio or the enantiomeric excess.

The term “enhancing” refers to increasing or improving a specific property.

The term “liver specificity” refers to the ratio:

[drug or a drug metabolite in liver tissue]

[drug or a drug metabolite in blood or another tissue]

as measured in animals treated with the drug or a prodrug. The ratio can be determined by measuring tissue levels at a specific time or may represent an AUC based on values measured at three or more time points.

The term “increased or enhanced liver specificity” refers to an increase in the liver specificity ratio in animals treated with the prodrug relative to animals treated with the parent drug.

The term “enhanced oral bioavailability” refers to an increase of at least 50% of the absorption of the dose of the parent drug. In an additional aspect the increase in oral bioavailability of the prodrug (compared to the parent drug) is at least 100%, that is a doubling of the absorption. Measurement of oral bioavailability usually refers to measurements of the prodrug, drug, or drug metabolite in blood, plasma, tissues, or urine following oral administration compared to measurements following parenteral administration.

The term “therapeutic index” refers to the ratio of the dose of a drug or prodrug that produces a therapeutically beneficial response relative to the dose that produces an undesired response such as death, an elevation of markers that are indicative of toxicity, and/or pharmacological side effects.

The term “sustained delivery” refers to an increase in the period in which there is a prolongation of therapeutically-effective drug levels due to the presence of the prodrug.

The term “bypassing drug resistance” refers to the loss or partial loss of therapeutic effectiveness of a drug (drug resistance) due to changes in the biochemical pathways and cellular activities important for producing and maintaining the biological activity of the drug and the ability of an agent to bypass this resistance through the use of alternative pathways or the failure of the agent to induce changes that tend to resistance.

The terms “treating” or “treatment” of a disease includes inhibiting the disease (slowing or arresting its development), providing relief from the symptoms or side-effects of the disease (including palliative treatment), and relieving the disease (causing regression of the disease).

Compounds provided herein are administered in a total daily dose of 0.01 to 1000 mg/kg. In some embodiments, the range is about 0.1 mg/kg to about 100 mg/kg. In some embodiments, the range is 0.5 to 20 mg/kg. The dose may be administered in as many divided doses as is convenient.

Compounds provided herein, when used in combination with other antiviral agents may be administered as a daily dose or an appropriate fraction of the daily dose (e.g., bid). Administration of the prodrug may occur at or near the time in which the other antiviral is administered or at a different time. The compounds provided herein may be used in a multidrug regimen, also known as combination or ‘cocktail’ therapy, wherein, multiple agents may be administered together, may be administered separately at the same time or at different intervals, or administered sequentially. The compounds provided herein may be administered after a course of treatment by another agent, during a course of therapy with another agent, administered as part of a therapeutic regimen, or may be administered prior to therapy by another agent in a treatment program.

The compounds provided herein may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters. Intravenous administration is generally preferred.

Pharmaceutically acceptable salts include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the compounds provided herein contain the active materials in admixture with excipients suitable for the manufacture of aqueous suspensions. Such excipients include a suspending agent, such as sodium carboxymethylcellulose, methylcellulose, ethylcellulose, hydroxypropylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachid oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules provided herein, suitable for preparation of an aqueous suspension by the addition of water, provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions provided herein may also be in the form of oil-in-water emulsions. The oily phase may be a vegetable oil, such as olive oil or arachid oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions provided herein may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain 20 to 2000 μmol (approximately 10 to 1000 mg) of active material compounded with an appropriate and convenient amount of carrier material which may vary from about 5 to about 95% of the total compositions. In some embodiments the pharmaceutical composition is prepared which provides easily measurable amounts for administration. For example, an aqueous solution intended for intravenous infusion should contain from about 0.05 to about 50 μmol (approximately 0.025 to 25 mg) of the active ingredient per milliliter of solution in order that infusion of a suitable volume at a rate of about 30 mL/h can occur.

As noted above, formulations of the compounds provided herein suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of Formula I when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

Formulations suitable for parenteral administration may be administered in a continuous infusion manner via an indwelling pump or via a hospital bag. Continuous infusion includes the infusion by an external pump. The infusions may be done through a Hickman or PICC or any other suitable means of administering a formulation either parenterally or i.v.

Preferred unit dosage formulations are those containing a daily dose or unit, daily sub-dose, or an appropriate fraction thereof, of a drug.

It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

Some embodiments are concerned with a method of inhibiting HCV replication or treating HCV infection with a compound provided herein in combination with one or more agents useful for treating HCV infection. Such agents active against HCV include, but are not limited to, ribavirin, levovirin, viramidine, thymosin alpha-1, interferon-β, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), pegylated interferon-α2a (Pegasys™), interferon-α2b (such as Intron-A interferon available from Schering Corp., Kenilworth, N.J.), pegylated interferon-α2b (PegIntron™), a recombinant consensus interferon (such as interferon alphacon-1), and a purified interferon-α product. Amgen's recombinant consensus interferon has the brand name Infergen®. Levovirin is the L-enantiomer of ribavirin which has shown immunomodulatory activity similar to ribavirin. Viramidine is a liver-targeting prodrug analog of ribavirin disclosed in WO 01/60379 (assigned to ICN Pharmaceuticals). In accordance with this method, the individual components of the combination can be administered separately at different times during the course of therapy or concurrently in divided or single combination forms. Some embodiments are therefore to be understood as embracing all such regimes of simultaneous or alternating treatment, and the term “administering” is to be interpreted accordingly. It will be understood that the scope of combinations of the compounds provided herein with other agents useful for treating HCV infection includes in principle any combination with any pharmaceutical composition for treating HCV infection. When a compound provided herein or a pharmaceutically acceptable salt thereof is used in combination with a second therapeutic agent active against HCV, the dose of each compound may be either the same as or different from the dose when the compound is used alone.

Some embodiments include a pharmaceutical composition comprising a compound of Formula I or II or pharmaceutically acceptable salt thereof and at least one agent useful for treating a viral infection, particularly an HCV infection.

For the treatment of HCV infection, the compounds provided herein may also be administered in combination with an agent that is an inhibitor of HCV NS3 serine protease. HCV NS3 serine protease is an essential viral enzyme and has been described to be an excellent target for inhibition of HCV replication. Both substrate and non-substrate based inhibitors of HCV NS3 protease inhibitors are disclosed in WO 98/22496, WO 98/46630, WO 99/07733, WO 99/07734, WO 99/38888, WO 99/50230, WO 99/64442, WO 00/09543, WO 00/59929, GB-2337262, WO 02/48116, WO 02/48172, U.S. Pat. No. 6,323,180, and U.S. Pat. No. 6,410,531. Specific embodiments of NS3 protease inhibitors for combination with the compounds provided herein are BILN 2061 (Boehringer Ingelheim) and VX-950/LY-570310. HCV NS3 protease as a target for the development of inhibitors of HCV replication and for the treatment of HCV infection is discussed in B. W. Dymock, “Emerging therapies for hepatitis C virus infection,” Emerging Drugs, 6: 13-42 (2001).

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of HCV replication. Therefore, the compounds provided herein may also be administered in combination with an inhibitor of IMPDH, such as VX-497 (merimepodib), which is disclosed in WO 97/41211 and WO 01/00622 (assigned to Vertex); another IMPDH inhibitor, such as that disclosed in WO 00/25780 (assigned to Bristol-Myers Squibb); or mycophenolate mofetil [see A. C. Allison and E. M. Eugui, Agents Action, 44 (Suppl.): 165 (1993)].

For the treatment of HCV infection, the compounds provided herein may also be administered in combination with the antiviral agent amantadine (1-aminoadamantane) and its hydrochloride salt [for a comprehensive description of this agent, see J. Kirschbaum, Anal. Profiles Drug Subs. 12: 1-36 (1983)].

The compounds provided herein may also be combined for the treatment of HCV infection with antiviral 1′-C, 2′-C-, or 3′-C-branched ribonucleosides disclosed in R. E. Harry-O'kuru, et al., J. Org. Chem., 62: 1754-1759 (1997); M. S. Wolfe, et al., Tetrahedron Lett., 36: 7611-7614 (1995); U.S. Pat. No. 3,480,613 (Nov. 25, 1969); International Publication Number WO 01/90121 (29 Nov. 2001); International Publication Number WO 01/92282 (6 Dec. 2001); and International Publication Number WO 02/32920 (25 Apr. 2002); the contents of each of which are incorporated by reference in their entirety. Such branched ribonucleosides include, but are not limited to, 2′-C-methylcytidine, 2′-C-methyluridine, 2′-C-methyladenosine, 2′-C-methylguanosine, and 9-(2-C-methyl-(3-D-ribofuranosyl)-2,6-diaminopurine, and prodrugs thereof.

The compounds provided herein may also be combined for the treatment of HCV infection with other nucleosides having anti-HCV properties, such as those disclosed in WO 02/51425 (4 Jul. 2002), assigned to Mitsubishi Pharma Corp.; WO 01/79246, WO 02/32920 (25 Apr. 2002), and WO 02/48165 (20 Jun. 2002), assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), assigned to Hoffmann-LaRoche; U.S. 2002/0019363 (14 Feb. 2002); WO 02/057287 (25 Jul. 2002), assigned to Merck & Co. and Isis Pharmaceuticals; and WO 02/057425 (25 Jul. 2002), assigned to Merck & Co. and Isis Pharmaceuticals.

The compounds provided herein may also be combined for the treatment of HCV infection with non-nucleoside inhibitors of HCV polymerase such as those disclosed in WO 01/77091 (18 Oct. 2001), assigned to Tularik, Inc.; WO 01/47883 (5 Jul. 2001), assigned to Japan Tobacco, Inc.; WO 02/04425 (17 Jan. 2002), assigned to Boehringer Ingelheim; WO 02/06246 (24 Jan. 2002), assigned to Istituto di Ricerche di Biologia Moleculare P. Angeletti S. P. A.; and WO 02/20497 (3 Mar. 2002). WO 01/47883 discloses a large number of benzimidazole derivatives, such as JTK-003, which is claimed to be an orally active inhibitor of NS5B that is currently undergoing clinical evaluation.

### Certain Synthesis Methods

Synthesis of the 5′-nucleoside monophosphate (NMP) prodrugs described herein is organized into two sections: 1. synthesis of phosphorylation precursors; 2. synthesis of prodrugs via coupling of nucleosides and prodrug moiety. General synthesis of the compounds of liver-targeting nucleoside prodrug derivatives has been discussed in detail previously (U.S. Pat. No. 7,666,855).

Scheme I describes general strategies of synthesis of the 2′-methyl nucleoside analogs. The first strategy starts with protection of the 3′-hydroxy group of nucleosides of structure 1 to generate intermediates of structure 2. The phosphate group is introduced by reaction of compounds of structure 2 and a reagent of structure 3 to give the monophosphate compounds of structure 4 following a deprotection of the 3′-hydroxy group. Treatment of compounds of structure 4 with a reagent of structure 5 provides the final compounds of structure 6. Alternatively, nucleoside of structure 1 can be phosphorylated directly with reagent of structure 3 without protection of the 3′-hydroxy group and then be acylated to afford the final compounds of structure 6. The third strategy is to prepare the intermediates of structure 7 and then is converted to final compounds of structure 6 by treatment with reagent of structure 3.

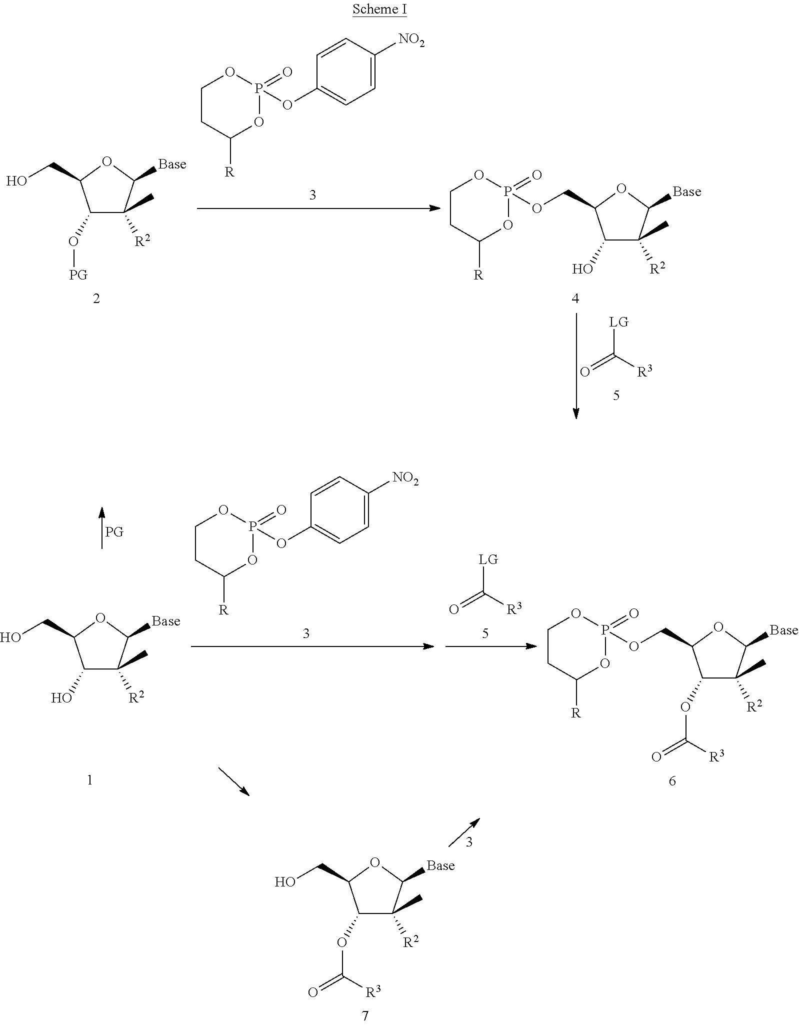

### EXAMPLES

Some compounds of Formula I and II are prepared as outlined below.

### Example 1

### 2(R)-(2-Amino-6-hydroxy-9(H)-purinyl)-4(R)-(4(S)-(3,5-difluorophenyl)-2-oxo-1,3,2-dioxaphosphorinan-2-(R)-yloxymethyl)-1(R)-methyl-7-oxo-3,6,8-trioxa-cis-bicyclo[3.3.0]octane (Compound 101)

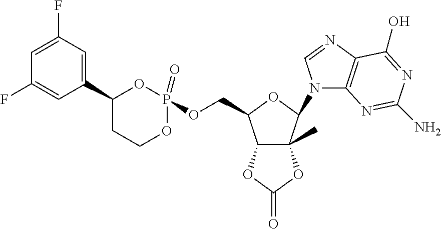 [SMILES: C[C@@]12OC(=O)O[C@@H]1[C@@H](CO[P@@]1(=O)OCC[C@@H](c3cc(F)cc(F)c3)O1)O[C@H]2n1cnc2c(O)nc(N)nc21]

Compound 101 was prepared according synthetic strategy of Scheme I from 2′-β-C-methylguanosine:

2(R)-(2-Amino-6-hydroxy-9(H)-purinyl)-4(R)-tert-butyldiphenylsilyloxymethyl-1(R)-methyl-7-oxo-3,6,8-trioxa-cis-bicyclo[3.3.0]octane: To a suspension of 2′-β-C-methylguanosine (0.5 g, 1.7 mmol) in DMF (8.3 mL) was added TBDPSCl (0.46 mL, 1.8 mmol) and imidazole (330 mg, 4.9 mmol), and the resulting mixture was stirred at room temperature for 30 min. 1,1′-Carbonyldiimidazole (530 mg, 3.3 mmol) was then added and the reaction mixture was stirred for several hours till the reaction was complete monitored by TLC. The reaction was diluted by ethyl acetate, washed with saturated NH₄Cl aqueous solution, and concentrated to provide crude product as off-white solid. The crude was suspended in dichloromethane and filtered to give the final compound (640 mg, 68%).

2(R)-(2-Amino-6-hydroxy-9(H)-purinyl)-4(R)-hydroxymethyl-1(R)-methyl-7-oxo-3,6,8-trioxa-cis-bicyclo[3.3.0]octane: To a solution of the TBDOS-protected compound (0.63 g, 1.1 mmol) in THF (6 mL) was added TEAF (0.38 g, 2.2 mmol) and acetic acid (0.13 mL, 2.2 mmol), and the reaction was stirred at room temperature for several hours to give large amount precipitate. The crude mixture was concentrated and then washed with methanol and DCM to afford the final compound (0.3 g, 83%).

Compound 101: To a suspension of the above guanosine derivative (0.3 g, 0.93 mmol) in DMF (10 mL) at 0° C. was added t-BuMgCl 2.0 M ether solution (0.83 mL, 1.6 mmol) and then 2(S)-(4-nitrophenoxy)-4(S)-(3,5-difluorophenyl)-2-oxo-1,3,2-dioxaphosphorinane (0.41 g, 1.1 mmol) to give a clear yellow solution. The reaction was stirred at 0° C. for several hours till completion by LC-MS and was quenched with aqueous saturated KH₂PO₄ solution (˜50 mL). The reaction mixture was diluted in ethyl acetate and water, and then extracted by ethyl acetate. Standard washing, drying, and concentration provided crude product that was taken into dichloromethane and filtered to afford compound 101 as white solid (0.19 g, 50% yield); mp 234-236° C.; MH⁺=556.6.

### Example 2

### 2(R)-(2-Amino-6-hydroxy-9 (H)-purinyl)-4(R)-(4(S)-(3-fluoro phenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 102) and 2(R)-(2-amino-6-hydroxy-9(H)-purinyl)-4(R)-(4(R)-(3-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 103)

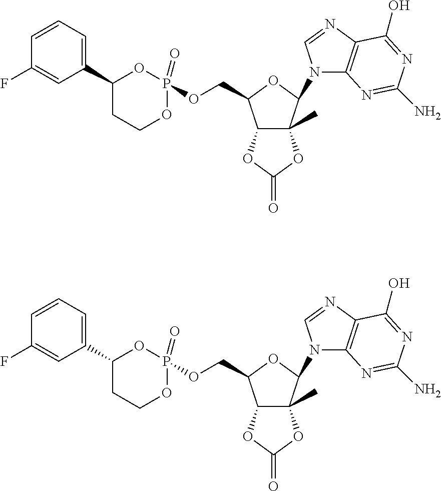 [SMILES: C[C@@]12OC(=O)O[C@@H]1[C@@H](CO[P@@]1(=O)OCC[C@@H](c3cccc(F)c3)O1)O[C@H]2n1cnc2c(O)nc(N)nc21 ; C[C@@]12OC(=O)O[C@@H]1[C@@H](CO[P@]1(=O)OCC[C@H](c3cccc(F)c3)O1)O[C@H]2n1cnc2c(O)nc(N)nc21]

Compounds 102 and 103 were prepared as a 1:1 mixture in a similar fashion as described in Example 1 by replacing 2(S)-(4-nitrophenoxy)-4(S)-(3,5-difluorophenyl)-2-oxo-1,3,2-dioxaphosphorinane with (±)-2-trans-(4-nitrophenoxy)-4-(3-fluorophenyl)-2-oxo-1,3,2-dioxaphosphorinane; mp 228-231° C.; MH+=538.4.

### Example 3

### 2(R)-(2-Amino-6-hydroxy-9 (H)-purinyl)-4(R)-(4(S)-(3-bromophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 104) and 2(R)-(2-amino-6-hydroxy-9(H)-purinyl)-4(R)-(4(R)-(3-bromo phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 105)

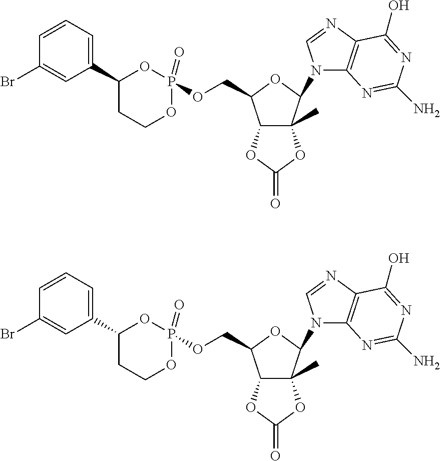 [SMILES: C[C@@]12OC(=O)O[C@@H]1[C@@H](CO[P@@]1(=O)OCC[C@@H](c3cccc(Br)c3)O1)O[C@H]2n1cnc2c(O)nc(N)nc21 ; C[C@@]12OC(=O)O[C@@H]1[C@@H](CO[P@]1(=O)OCC[C@H](c3cccc(Br)c3)O1)O[C@H]2n1cnc2c(O)nc(N)nc21]

Compounds 104 and 105 were prepared as a 1:1 mixture in a similar fashion as described in Example 1 by replacing 2(S)-(4-nitrophenoxy)-4(S)-(3,5-difluorophenyl)-2-oxo-1,3,2-dioxaphosphorinane with (±)-2-trans-(4-nitrophenoxy)-4-(3-bromophenyl)-2-oxo-1,3,2-dioxaphosphorinane; mp 232-235° C.; MH⁺=600.4.

### Example 4

### 2(R)-(2-Amino-6-hydroxy-9 (H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 106)

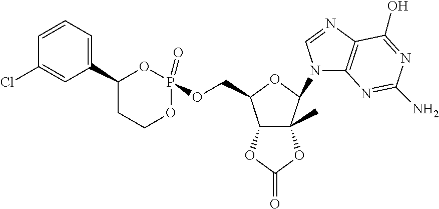 [SMILES: C[C@@]12OC(=O)O[C@@H]1[C@@H](CO[P@@]1(=O)OCC[C@@H](c3cccc(Cl)c3)O1)O[C@H]2n1cnc2c(O)nc(N)nc21]

Compound 106 was prepared in a similar fashion as described in Example 1 by replacing 2(S)-(4-nitrophenoxy)-4(S)-(3,5-difluorophenyl)-2-oxo-1,3,2-dioxaphosphorinane with 2(S)-(4-nitrophenoxy)-4(S)-(3-chlorophenyl)-2-oxo-1,3,2-dioxaphosphorinane; mp>200° C.; MH⁺=554.6.

### Example 5

### 2(R)-(2-Amino-6-hydroxy-9(H)-purinyl)-4(R)-(4(S)-(2-bromophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 107) and 2(R)-(2-amino-6-hydroxy-9(H)-purinyl)-4(R)-(4(R)-(2-bromo phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 108)

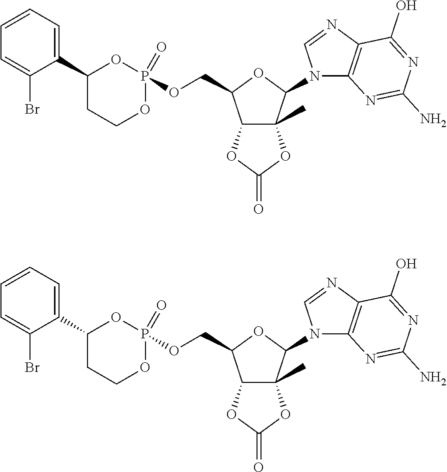 [SMILES: C[C@@]12OC(=O)O[C@@H]1[C@@H](CO[P@@]1(=O)OCC[C@@H](c3ccccc3Br)O1)O[C@H]2n1cnc2c(O)nc(N)nc21 ; C[C@@]12OC(=O)O[C@@H]1[C@@H](CO[P@]1(=O)OCC[C@H](c3ccccc3Br)O1)O[C@H]2n1cnc2c(O)nc(N)nc21]

Compounds 107 and 108 were prepared as a 1:1 mixture in a similar fashion as described in Example 1 by replacing 2(S)-(4-nitrophenoxy)-4(S)-(3,5-difluorophenyl)-2-oxo-1,3,2-dioxaphosphorinane with (±)-2-trans-(4-nitrophenoxy)-4-(2-bromophenyl)-2-oxo-1,3,2-dioxaphosphorinane; mp 215-220° C.; MH⁺=554.6.

### Example 6

### 2(R)-(2-Amino-6-hydroxy-9 (H)-purinyl)-4(R)-(4(S)-(3-cyanophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 109)

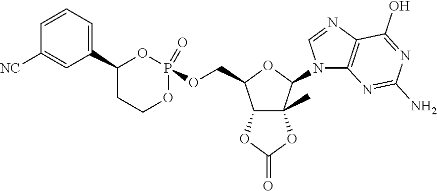 [SMILES: C[C@@]12OC(=O)O[C@@H]1[C@@H](CO[P@@]1(=O)OCC[C@@H](c3cccc(C#N)c3)O1)O[C@H]2n1cnc2c(O)nc(N)nc21]

Compound 109 was prepared in a similar fashion as described in Example 1 by replacing 2(S)-(4-nitrophenoxy)-4(S)-(3,5-difluorophenyl)-2-oxo-1,3,2-dioxaphosphorinane with 2(S)-(4-nitro phenoxy)-4(S)-(3-cyanophenyl)-2-oxo-1,3,2-dioxaphosphorinane; mp 218-220° C.; MH⁺=545.4.

### Example 7

### 2(R)-(2-Amino-6-hydroxy-9(H)-purinyl)-4(R)-(4(S)-(2-pyridyl)-2-oxo-1,3,2-dioxaphosphorinan-2(R)-yloxymethyl)-1(R)-methyl-7-oxo-3,6,8-trioxa-cis-bicyclo[3.3.0]octane (Compound 110) and 2(R)-(2-amino-6-hydroxy-9(H)-purinyl)-4(R)-(4(R)-(2-pyridyl)-2-oxo-1,3,2-dioxaphosphorinan-2(S)-yloxymethyl)-1(R)-methyl-7-oxo-3,6,8-trioxa-cis-bicyclo[3.3.0]octane (Compound III)

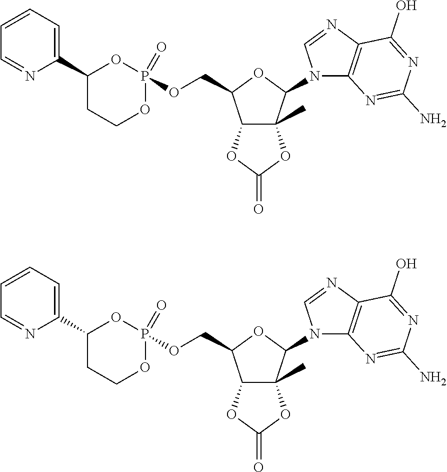 [SMILES: C[C@@]12OC(=O)O[C@@H]1[C@@H](CO[P@@]1(=O)OCC[C@@H](c3ccccn3)O1)O[C@H]2n1cnc2c(O)nc(N)nc21 ; C[C@@]12OC(=O)O[C@@H]1[C@@H](CO[P@]1(=O)OCC[C@H](c3ccccn3)O1)O[C@H]2n1cnc2c(O)nc(N)nc21]

Compounds 110 and 111 were prepared as a 1:1 mixture in a similar fashion as described in Example 1 by replacing 2(S)-(4-nitrophenoxy)-4(S)-(3,5-difluorophenyl)-2-oxo-1,3,2-dioxaphosphorinane with (±)-2-trans-(4-nitrophenoxy)-4-(2-pyridyl)-2-oxo-1,3,2-dioxaphosphorinane and were isolated as a trifluoroacetate salt; mp 185-187° C.; MH⁺=521.4.

### Example 8

### 2(R)-(2-Amino-6-hydroxy-9 (H)-purinyl)-4(R)-(4(S)-(3-pyridyl)-2-oxo-1,3,2-dioxaphosphorinan-2(R)-yloxymethyl)-1(R)-methyl-7-oxo-3,6,8-trioxa-cis-bicyclo[3.3.0]octane (Compound 112) and 2(R)-(2-amino-6-hydroxy-9(H)-purinyl)-4(R)-(4(R)-(3-pyridyl)-2-oxo-1,3,2-dioxaphosphorinan-2(S)-yloxymethyl)-1(R)-methyl-7-oxo-3,6,8-trioxa-cis-bicyclo[3.3.0]octane (Compound 113)

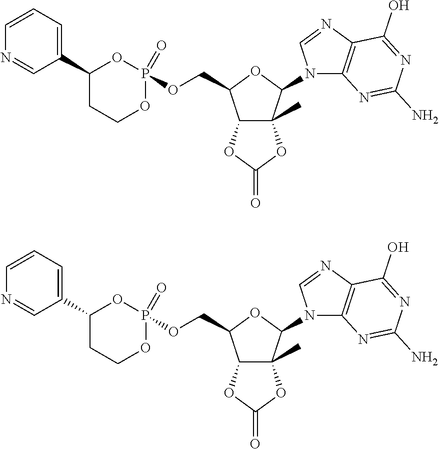 [SMILES: C[C@@]12OC(=O)O[C@@H]1[C@@H](CO[P@@]1(=O)OCC[C@@H](c3cccnc3)O1)O[C@H]2n1cnc2c(O)nc(N)nc21 ; C[C@@]12OC(=O)O[C@@H]1[C@@H](CO[P@]1(=O)OCC[C@H](c3cccnc3)O1)O[C@H]2n1cnc2c(O)nc(N)nc21]

Compounds 112 and 113 were prepared as a 1:1 mixture in a similar fashion as described in Example 1 by replacing 2(S)-(4-nitrophenoxy)-4(S)-(3,5-difluorophenyl)-2-oxo-1,3,2-dioxaphosphorinane with (±)-2-trans-(4-nitrophenoxy)-4-(3-pyridyl)-2-oxo-1,3,2-dioxaphosphorinane and were isolated as a white trifluoroacetate salt; mp 205 (dec)° C.; MH⁺=521.3.

### Example 9

### 2(R)-(2-Amino-6-hydroxy-9(H)-purinyl)-4(R)-(4(S)-(3,5-dichlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 114) and 2(R)-(2-amino-6-hydroxy-9(H)-purinyl)-4(R)-(4(R)-(3,5-dichlorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 115)

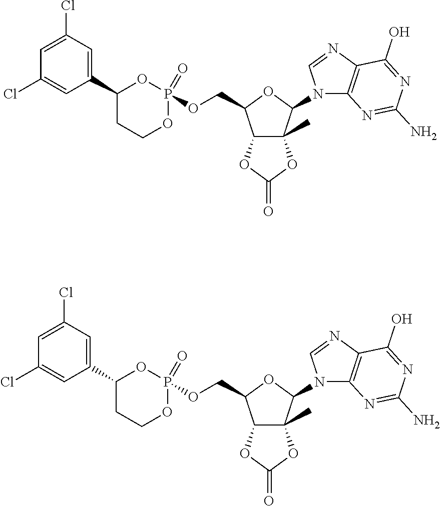 [SMILES: C[C@@]12OC(=O)O[C@@H]1[C@@H](CO[P@@]1(=O)OCC[C@@H](c3cc(Cl)cc(Cl)c3)O1)O[C@H]2n1cnc2c(O)nc(N)nc21 ; C[C@@]12OC(=O)O[C@@H]1[C@@H](CO[P@]1(=O)OCC[C@H](c3cc(Cl)cc(Cl)c3)O1)O[C@H]2n1cnc2c(O)nc(N)nc21]

Compounds 114 and 115 were prepared as a 1:1 mixture in a similar fashion as described in Example 1 by replacing 2(S)-(4-nitrophenoxy)-4(S)-(3,5-difluorophenyl)-2-oxo-1,3,2-dioxaphosphorinane with (±)-2-trans-(4-nitrophenoxy)-4-(3,5-dichlorophenyl)-2-oxo-1,3,2-dioxaphosphorinane and were isolated as a white trifluoroacetate salt; mp 190 (dec)° C.; MH⁺=588.2.

### Example 10

### 2(R)-(2-Amino-6-hydroxy-9(H)-purinyl)-4(R)-(4(S)-(4-pyridyl)-2-oxo-1,3,2-dioxaphosphorinan-2(R)-yloxymethyl)-1(R)-methyl-7-oxo-3,6,8-trioxa-cis-bicyclo[3.3.0]octane (Compound 116)

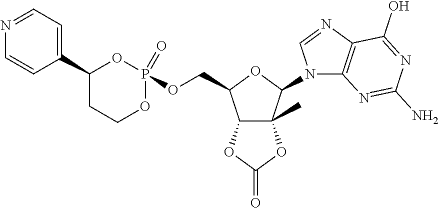 [SMILES: C[C@@]12OC(=O)O[C@@H]1[C@@H](CO[P@@]1(=O)OCC[C@@H](c3ccncc3)O1)O[C@H]2n1cnc2c(O)nc(N)nc21]

Compound 116 was prepared in a similar fashion as described in Example 1 by replacing 2(S)-(4-nitrophenoxy)-4(S)-(3,5-difluorophenyl)-2-oxo-1,3,2-dioxaphosphorinane with 2(S)-(4-nitrophenoxy)-4(S)-(4-pyridyl)-2-oxo-1,3,2-dioxaphosphorinane; mp 230 (Dec)° C.; MH⁺=521.3.

### Example 11

### 2(R)-(2-Amino-6-hydroxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-(2-methyl-1-oxopropoxy)-tetrahydrofuran (Compound 117)

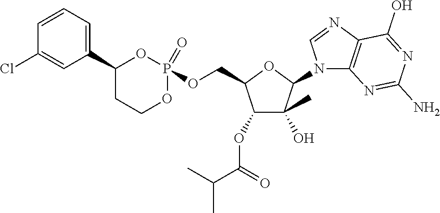 [SMILES: CC(C)C(=O)O[C@@H]1[C@@H](CO[P@@]2(=O)OCC[C@@H](c3cccc(Cl)c3)O2)O[C@@H](n2cnc3c(O)nc(N)nc32)[C@]1(C)O]

Compound 117 was prepared according to the strategy described in Scheme I. Briefly, to a solution of 2(R)-(2-amino-6-hydroxy-9(H)-purinyl)-5(R)-(4(S)-(3-chlorophenyl)-2-oxo-1,3,2-dioxaphosphorinan-2(R)-yloxymethyl)-3(R),4(R)-dihydroxy-3(R)-methyltetrahydrofuran (0.26 g, 0.5 mmol) in pyridine (5 mL) was added 4-DMAP (6 mg, 0.05 mmol) and isobutyric anhydride (0.1 mL, 0.5 mmol), and the reaction mixture was stirred at room temperature overnight. Standard work-up procedure followed by HPLC provided compound 117 as a white powder; mp>200° C.; MH⁺=598.6.

### Example 12

### 2(R)-(2-Amino-6-hydroxy-9 (H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-L-valinyl-tetrahydrofuran (Compound 118)

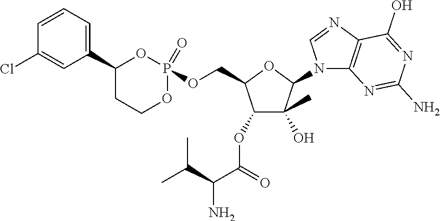 [SMILES: CC(C)[C@H](N)C(=O)O[C@@H]1[C@@H](CO[P@@]2(=O)OCC[C@@H](c3cccc(Cl)c3)O2)O[C@@H](n2cnc3c(O)nc(N)nc32)[C@]1(C)O]

Compound 118 was prepared in a similar fashion as described in Example 11 by replacing isobutyric anhydride with activated L-valine. Compound 118 was isolated as a off-white trifluoroacetate salt; mp>210° C.; MH⁺=627.6.

### Example 13

### 2(R)-(2-Amino-6-methoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 119)

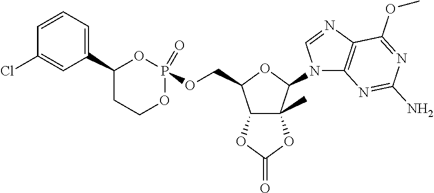 [SMILES: COc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 119 was prepared in a similar fashion as described in Example 1 starting from 6-O-methyl-2′-β-C-methylguanosine.

2(R)-(2-Amino-6-methoxy-9(H)-purinyl)-5(R)-(4(S)-(3-chlorophenyl)-2-oxo-1,3,2-dioxaphosphorinan-2(R)-yloxymethyl)-3(R),4(R)-dihydroxy-3(R)-methyltetrahydrofuran: To a solution of 6-O-methyl-2′-β-C-methylguanosine (1.6 g, 5.2 mmol) in DMF (42 mL) at 0° C. was added dropwise a 1.0 M t-BuMgCl THF solution (13 mL, 13 mmol). The resulting mixture was stirred for 20 min and then was added 2(S)-(4-nitrophenoxy)-4(S)-(3-cholorophenyl)-2-oxo-1,3,2-dioxaphosphorinane (2.3 g, 6.2 mmol). The reaction was allowed to warm up to room temperature and then quenched with saturated aqueous KH₂PO₄ (50 mL). Standard work-up followed by chromatography gave the product (0.78 g) as a white solid; mp 178-184° C.; MH⁺=542.6.

Compound 119: To a solution of the above compound (0.78 g, 1.4 mmol) in DMF (15 mL) at room temperature under nitrogen was added CDI (0.7 g, 4.3 mmol) and the resulting mixture was stirred for several hours till the reaction went completion by TLC. Standard work-up followed by chromatography provided compound 119 (0.55 g, 67%) as a white solid; mp 125-130° C.; MH⁺=568.6.

### Example 14

### 2(R)-(2-Amino-6-ethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 120)

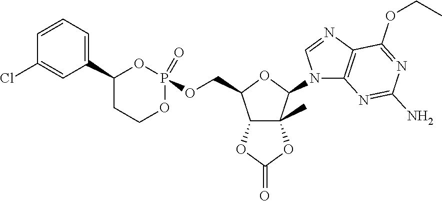 [SMILES: CCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 120 was prepared in a similar fashion as described in Example 13 starting from 6-O-ethyl-2′-β-C-methylguanosine instead of 6-O-methyl-2′-β-C-methylguanosine; mp 82-84° C.; MH⁺=582.3.

### Example 15

### 2(R)-(2-Amino-6-cyclopropylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 121)

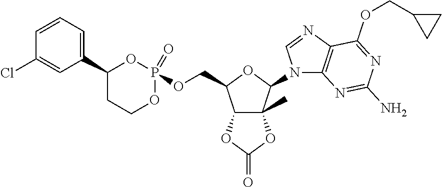 [SMILES: C[C@@]12OC(=O)O[C@@H]1[C@@H](CO[P@@]1(=O)OCC[C@@H](c3cccc(Cl)c3)O1)O[C@H]2n1cnc2c(OCC3CC3)nc(N)nc21]

Compound 121 was prepared in a similar fashion as described in Example 13 starting from 6-O-cyclopropylmethyl-2′-β-C-methylguanosine instead of 6-O-methyl-2′-β-C-methylguanosine; mp 128-132° C.; MH⁺=608.6.

### Example 16

### 2(R)-(2-Amino-6-(2-methylpropyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 122)

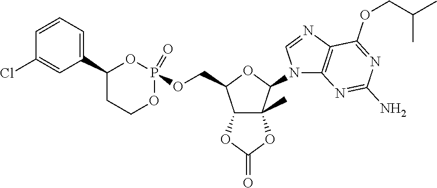 [SMILES: CC(C)COc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 122 was prepared in a similar fashion as described in Example 13 starting from 6-O-(2-methylpropyl)-2′-β-C-methylguanosine instead of 6-O-methyl-2′-β-C-methylguanosine; mp 118-122° C.; MH⁺=610.6.

### Example 17

### 2(R)-(2-Amino-6-ethoxy-9(H)-purinyl)-4(R)-(4(S)-(3-bromophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 123)

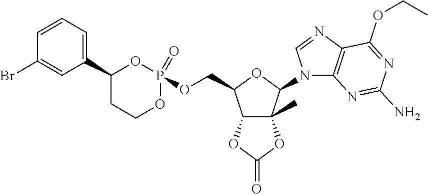 [SMILES: CCOc1nc(N)nc2c1ncn2[C@@H]1O[C@H](CO[P@@]2(=O)OCC[C@@H](c3cccc(Br)c3)O2)[C@H]2OC(=O)O[C@]21C]

Compound 123 was prepared in a similar fashion as described in Example 14 by replacing 2(S)-(4-nitrophenoxy)-4(S)-(3-chlorophenyl)-2-oxo-1,3,2-dioxaphosphorinane with 2(S)-(4-nitro phenoxy)-4(S)-(3-bromophenyl)-2-oxo-1,3,2-dioxaphosphorinane; mp 77-80° C.; MH⁺=628.4.

### Example 18

### 2(R)-(2-Amino-6-ethoxy-9(H)-purinyl)-4(R)-(4(S)-(3-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 124)

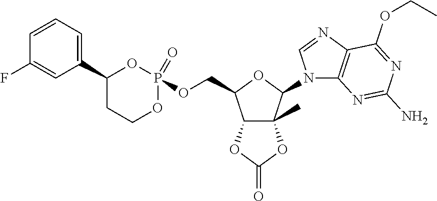 [SMILES: CCOc1nc(N)nc2c1ncn2[C@@H]1O[C@H](CO[P@@]2(=O)OCC[C@@H](c3cccc(F)c3)O2)[C@H]2OC(=O)O[C@]21C]

Compound 124 was prepared in a similar fashion as described in Example 14 by replacing 2(S)-(4-nitrophenoxy)-4(S)-(3-chlorophenyl)-2-oxo-1,3,2-dioxaphosphorinane with 2(S)-(4-nitrophenoxy)-4(S)-(3-fluorophenyl)-2-oxo-1,3,2-dioxaphosphorinane; mp 88-90° C.; MH⁺=566.4.

### Example 19

### 2(R)-(2-Amino-6-cyclopentyloxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 125)

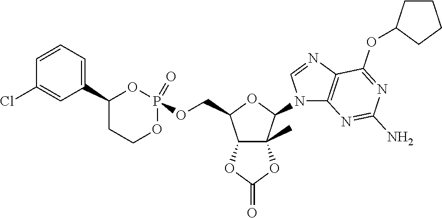 [SMILES: C[C@@]12OC(=O)O[C@@H]1[C@@H](CO[P@@]1(=O)OCC[C@@H](c3cccc(Cl)c3)O1)O[C@H]2n1cnc2c(OC3CCCC3)nc(N)nc21]

Compound 125 was prepared in a similar fashion as described in Example 13 starting from 6-O-(2-cyclopentyl)-2′-β-C-methylguanosine instead of 6-O-methyl-2′-β-C-methylguanosine; mp 107-109° C.; MH⁺=622.6.

### Example 20

### 2(R)-(2-Amino-6-ethoxy-9(H)-purinyl)-4(R)-(4(S)-(3,5-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 126)

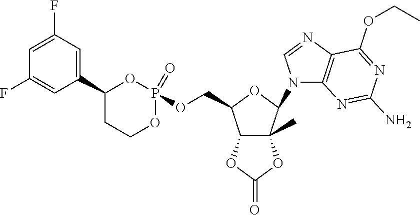 [SMILES: CCOc1nc(N)nc2c1ncn2[C@@H]1O[C@H](CO[P@@]2(=O)OCC[C@@H](c3cc(F)cc(F)c3)O2)[C@H]2OC(=O)O[C@]21C]

Compound 126 was prepared in a similar fashion as described in Example 14 by replacing 2(S)-(4-nitrophenoxy)-4(S)-(3-chlorophenyl)-2-oxo-1,3,2-dioxaphosphorinane with 2(S)-(4-nitrophenoxy)-4(S)-(3,5-difluorophenyl)-2-oxo-1,3,2-dioxaphosphorinane; mp 96-98° C.; MH⁺=584.4.

### Example 21

### 2(R)-(2-Amino-6-ethoxy-9(H)-purinyl)-4(R)-(4(S)-(3,5-dichlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 127)

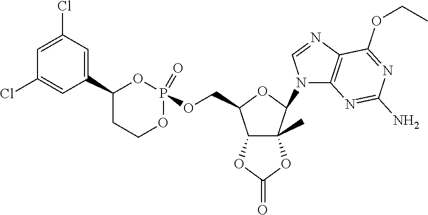 [SMILES: CCOc1nc(N)nc2c1ncn2[C@@H]1O[C@H](CO[P@@]2(=O)OCC[C@@H](c3cc(Cl)cc(Cl)c3)O2)[C@H]2OC(=O)O[C@]21C]

Compound 127 was prepared in a similar fashion as described in Example 14 by replacing 2(S)-(4-nitrophenoxy)-4(S)-(3-chlorophenyl)-2-oxo-1,3,2-dioxaphosphorinane with 2(S)-(4-nitro phenoxy)-4(S)-(3,5-dichlorophenyl)-2-oxo-1,3,2-dioxaphosphorinane; mp 101-103° C.; MH⁺=618.4.

### Example 22

### 2(R)-(2-Amino-6-ethoxy-9(H)-purinyl)-4(R)-(4(R)-(3,5-dichlorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 128)

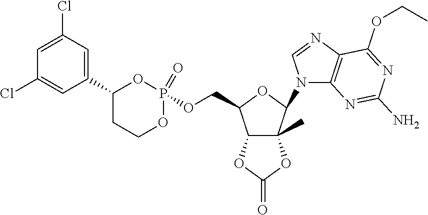 [SMILES: CCOc1nc(N)nc2c1ncn2[C@@H]1O[C@H](CO[P@]2(=O)OCC[C@H](c3cc(Cl)cc(Cl)c3)O2)[C@H]2OC(=O)O[C@]21C]

Compound 128 was prepared in a similar fashion as described in Example 14 by replacing 2(S)-(4-nitrophenoxy)-4(S)-(3-chlorophenyl)-2-oxo-1,3,2-dioxaphosphorinane with 2(R)-(4-nitrophenoxy)-4(R)-(3,5-dichlorophenyl)-2-oxo-1,3,2-dioxaphosphorinane; mp 101-103° C.; MH⁺=618.4.

### Example 23

### 2(R)-(2-Amino-6-(4-tetrahydropyranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 129)

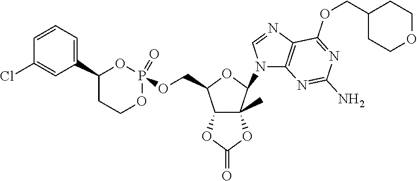 [SMILES: C[C@@]12OC(=O)O[C@@H]1[C@@H](CO[P@@]1(=O)OCC[C@@H](c3cccc(Cl)c3)O1)O[C@H]2n1cnc2c(OCC3CCOCC3)nc(N)nc21]

Compound 129 was prepared in a similar fashion as described in Example 13 starting from 6-O-(4-tetrahydropyranylmethyl)-2′-β-C-methylguanosine instead of 6-O-methyl-2′-β-C-methylguanosine; mp 105-108° C.; MH⁺=652.6.

### Example 24

### 2(R)-(2-Amino-6-(3-methylthiopropyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 130)

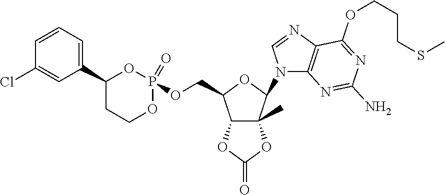 [SMILES: CSCCCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 130 was prepared in a similar fashion as described in Example 13 starting from 6-O-(3-methylthiopropyl)-2′-β-C-methylguanosine instead of 6-O-methyl-2′-β-C-methylguanosine; mp 79-82° C.; MH⁺=642.4.

### Example 25

### 2(R)-(2-Amino-6-(2-cyclohexylethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 131)

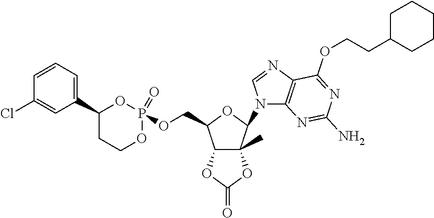 [SMILES: C[C@@]12OC(=O)O[C@@H]1[C@@H](CO[P@@]1(=O)OCC[C@@H](c3cccc(Cl)c3)O1)O[C@H]2n1cnc2c(OCCC3CCCCC3)nc(N)nc21]

Compound 131 was prepared in a similar fashion as described in Example 13 starting from 6-O-(2-cyclohexylethyl)-2′-β-C-methylguanosine instead of 6-O-methyl-2′-β-C-methylguanosine; mp 93-95° C.; MH⁺=664.6.

### Example 26

### 2(R)-(2-Amino-6-(3(R)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 132) and 2(R)-(2-amino-6-(3(S)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 133)

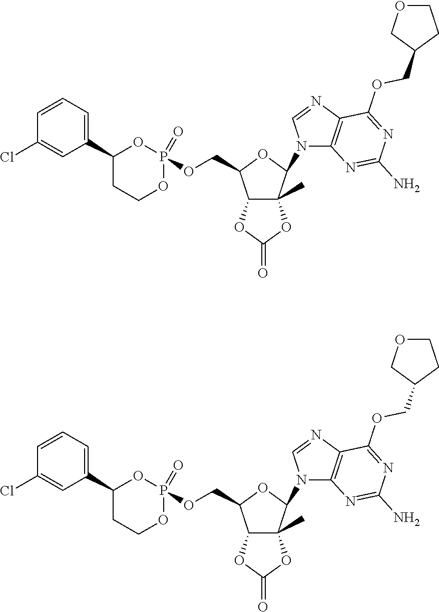 [SMILES: C[C@@]12OC(=O)O[C@@H]1[C@@H](CO[P@@]1(=O)OCC[C@@H](c3cccc(Cl)c3)O1)O[C@H]2n1cnc2c(OC[C@@H]3CCOC3)nc(N)nc21 ; C[C@@]12OC(=O)O[C@@H]1[C@@H](CO[P@@]1(=O)OCC[C@@H](c3cccc(Cl)c3)O1)O[C@H]2n1cnc2c(OC[C@H]3CCOC3)nc(N)nc21]

Compounds 132 and 133 were prepared as a 1:1 mixture in a similar fashion as described in Example 13 starting from a 1:1 mixture of 6-O-(3(R)-tetrahydrofuranylmethyl)-2′-β-C-methylguanosine and 6-O-(3(S)-tetrahydrofuranylmethyl)-2′-β-C-methylguanosine instead of 6-O-methyl-2′-β-C-methylguanosine; mp 105-108° C.; MH⁺=638.6.

### Example 27

### 2(R)-(2-Amino-6-(3-methylbutyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 134)

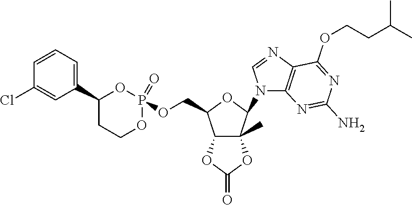 [SMILES: CC(C)CCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 134 was prepared in a similar fashion as described in Example 13 starting from 6-O-(3-methylbutyl)-2′-β-C-methylguanosine instead of 6-O-methyl-2′-β-C-methylguanosine; mp 76-78° C.; MH⁺=624.6.

### Example 28

### 2(R)-(2-Amino-6-(2-methylcyclopropylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 135)

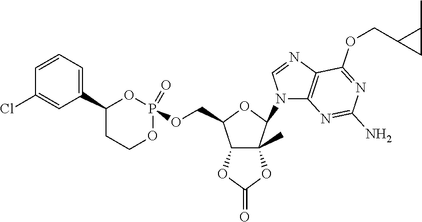 [SMILES: CC1CC1COc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 135 was prepared in a similar fashion as described in Example 13 starting from 6-O-(2-methylcyclopropylmethyl)-2′-β-C-methylguanosine instead of 6-O-methyl-2′-β-C-methylguanosine; mp 88-90° C.; MH⁺=622.6.

### Example 29

### 2(R)-(2-Amino-6-(2(R)-tetrahydropyranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 136) and 2(R)-(2-amino-6-(2(S)-tetrahydropyranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 137)

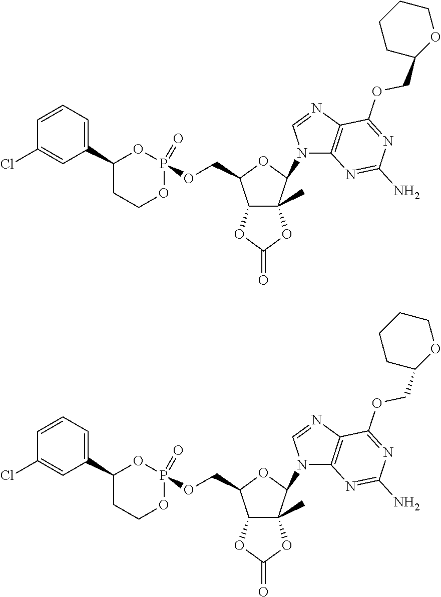 [SMILES: C[C@@]12OC(=O)O[C@@H]1[C@@H](CO[P@@]1(=O)OCC[C@@H](c3cccc(Cl)c3)O1)O[C@H]2n1cnc2c(OC[C@H]3CCCCO3)nc(N)nc21 ; C[C@@]12OC(=O)O[C@@H]1[C@@H](CO[P@@]1(=O)OCC[C@@H](c3cccc(Cl)c3)O1)O[C@H]2n1cnc2c(OC[C@@H]3CCCCO3)nc(N)nc21]

Compounds 136 and 137 were prepared as a 1:1 mixture in a similar fashion as described in Example 13 starting from a 1:1 mixture of 6-O-(2(R)-tetrahydropyranylmethyl)-2′-β-C-methylguanosine and 6-O-(2(S)-tetrahydropyranylmethyl)-2′-β-C-methylguanosine instead of 6-O-methyl-2′-β-C-methylguanosine; mp 135-138° C.; MH⁺=652.6.

### Example 30

### 2(R)-(2-Amino-6-(3-methyl-3-methoxybutyl oxy)-9 (H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 138)

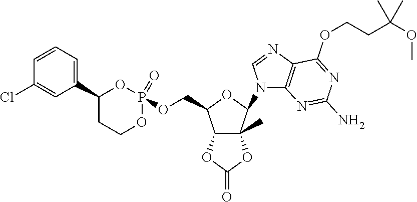 [SMILES: COC(C)(C)CCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 138 was prepared in a similar fashion as described in Example 13 starting from 6-O-(3-methyl-3-methoxybutyl)-2′-β-C-methylguanosine instead of 6-O-methyl-2′-β-C-methylguanosine; mp 93-96° C.; MH⁺=654.4.

### Example 31

### 2(R)-(2-Amino-6-(2-bicyclo[2.2.1]heptanylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 139)

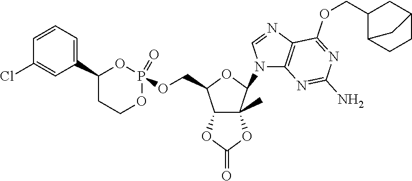 [SMILES: C[C@@]12OC(=O)O[C@@H]1[C@@H](CO[P@@]1(=O)OCC[C@@H](c3cccc(Cl)c3)O1)O[C@H]2n1cnc2c(OCC3CC4CCC3C4)nc(N)nc21]

Compound 139 was prepared in a similar fashion as described in Example 13 starting from 6-O-(2-bicyclo[2.2.1]heptanylmethyl)-2′-β-C-methylguanosine instead of 6-O-methyl-2′-β-C-methylguanosine; mp 99-101° C.; MH⁺=662.4.

### Example 32

### 2(R)-(2-Amino-6-cyclobutylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 140)

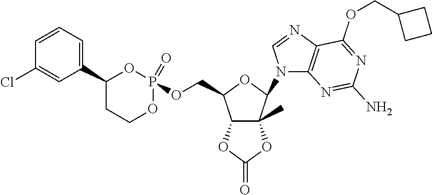 [SMILES: C[C@@]12OC(=O)O[C@@H]1[C@@H](CO[P@@]1(=O)OCC[C@@H](c3cccc(Cl)c3)O1)O[C@H]2n1cnc2c(OCC3CCC3)nc(N)nc21]

Compound 140 was prepared in a similar fashion as described in Example 13 starting from 6-O-cyclobutylmethyl-2′-β-C-methylguanosine instead of 6-O-methyl-2′-β-C-methylguanosine; mp 92-94° C.; MH⁺=622.6.

### Example 33

### 2(R)-(2-Amino-6-cyclopentylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 141)

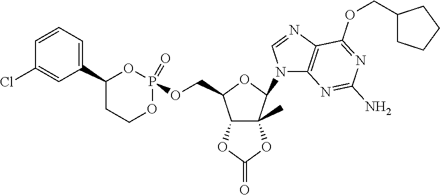 [SMILES: C[C@@]12OC(=O)O[C@@H]1[C@@H](CO[P@@]1(=O)OCC[C@@H](c3cccc(Cl)c3)O1)O[C@H]2n1cnc2c(OCC3CCCC3)nc(N)nc21]

Compound 141 was prepared in a similar fashion as described in Example 13 starting from 6-O-cyclopentylmethyl-2′-β-C-methylguanosine instead of 6-O-methyl-2′-β-C-methylguanosine; mp 99-101° C.; MH⁺=636.9.

### Example 34

### 2(R)-(2-Amino-6-(4-methyl-3(E)-pentenyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 142)

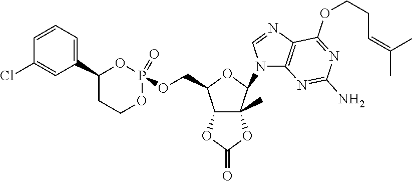 [SMILES: CC(C)=CCCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 142 was prepared in a similar fashion as described in Example 13 starting from 6-O-(4-methyl-3(E)-pentenyl)-2′-β-C-methylguanosine instead of 6-O-methyl-2′-β-C-methylguanosine; mp 72-75° C.; MH⁺=636.9.

### Example 35

### 2(R)-(2-Amino-6-(2-ethylbutyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 143)

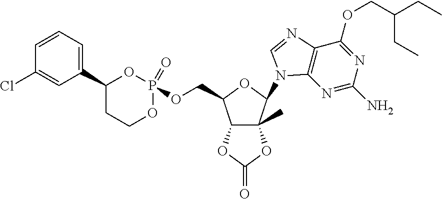 [SMILES: CCC(CC)COc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 143 was prepared in a similar fashion as described in Example 13 starting from 6-O-(4-methyl-3(E)-pentenyl)-2′-β-C-methylguanosine instead of 6-O-methyl-2′-β-C-methylguanosine; mp 72-75° C.; MH⁺=638.6.

### Example 36

### 2(R)-(2-Amino-6-(2(R)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 144) and 2(R)-(2-amino-6-(2(S)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 145)

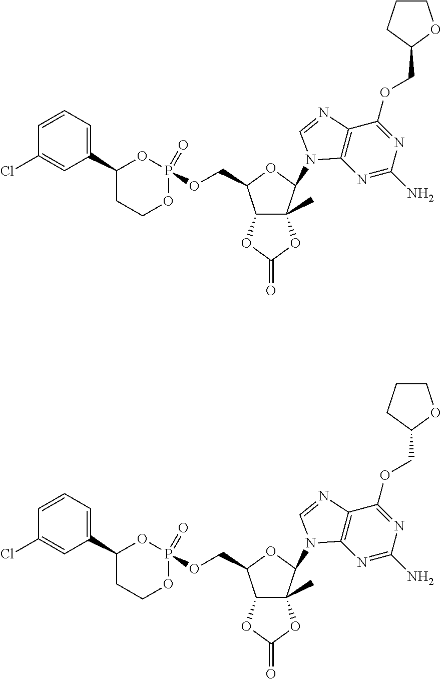 [SMILES: C[C@@]12OC(=O)O[C@@H]1[C@@H](CO[P@@]1(=O)OCC[C@@H](c3cccc(Cl)c3)O1)O[C@H]2n1cnc2c(OC[C@H]3CCCO3)nc(N)nc21 ; C[C@@]12OC(=O)O[C@@H]1[C@@H](CO[P@@]1(=O)OCC[C@@H](c3cccc(Cl)c3)O1)O[C@H]2n1cnc2c(OC[C@@H]3CCCO3)nc(N)nc21]

Compounds 144 and 145 were prepared as a 1:1 mixture in a similar fashion as described in Example 13 starting from a 1:1 mixture of 6-O-(2(R)-tetrahydrofuranylmethyl)-2′-β-C-methylguanosine and 6-O-(2(S)-tetrahydrofuranylmethyl)-2′-β-C-methylguanosine instead of 6-O-methyl-2′-β-C-methylguanosine; mp 92-95° C.; MH⁺=638.6.

### Example 37

### 2(R)-(2-Amino-6-cyclohexylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 146)

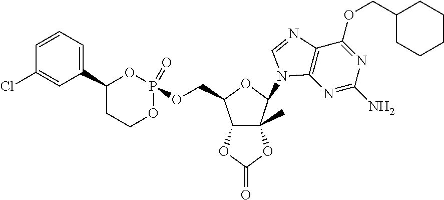 [SMILES: C[C@@]12OC(=O)O[C@@H]1[C@@H](CO[P@@]1(=O)OCC[C@@H](c3cccc(Cl)c3)O1)O[C@H]2n1cnc2c(OCC3CCCCC3)nc(N)nc21]

Compound 146 was prepared in a similar fashion as described in Example 13 starting from 6-O-cyclohexylmethyl-2′-β-C-methylguanosine instead of 6-O-methyl-2′-β-C-methylguanosine; mp 101-103° C.; MH⁺=650.6.

### Example 38

### 2(R)-(2-Amino-6-(2,2-dimethylpropyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 147)

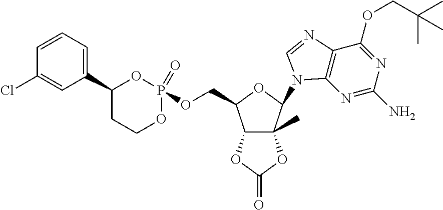 [SMILES: CC(C)(C)COc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 147 was prepared in a similar fashion as described in Example 13 starting from 6-O-(2,2-dimethylpropyl)-2′-β-C-methylguanosine instead of 6-O-methyl-2′-β-C-methylguanosine; mp 88-91° C.; MH⁺=624.6.

### Example 39

### 2(R)-(2-Amino-6-(2-ethoxyethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 148)

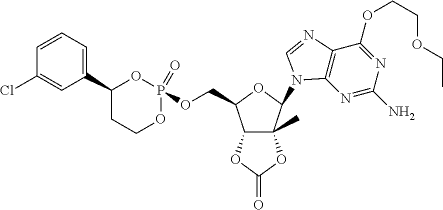 [SMILES: CCOCCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 148 was prepared in a similar fashion as described in Example 13 starting from 6-O-(2-ethoxyethyl)-2′-β-C-methylguanosine instead of 6-O-methyl-2′-β-C-methylguanosine; mp 170-103° C.; MH⁺=626.6.

### Example 40

### 2(R)-(2-Amino-6-(2,2-diethoxyethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 149)

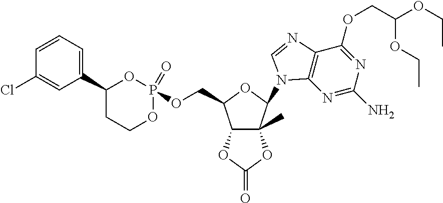 [SMILES: CCOC(COc1nc(N)nc2c1ncn2[C@@H]1O[C@H](CO[P@@]2(=O)OCC[C@@H](c3cccc(Cl)c3)O2)[C@H]2OC(=O)O[C@]21C)OCC]

Compound 149 was prepared in a similar fashion as described in Example 13 starting from 6-O-(2,2-diethoxyethyl)-2′-β-C-methylguanosine instead of 6-O-methyl-2′-β-C-methylguanosine; mp 72-75° C.

### Example 41

### 2(R)-(2-Amino-6-pentyloxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 150)

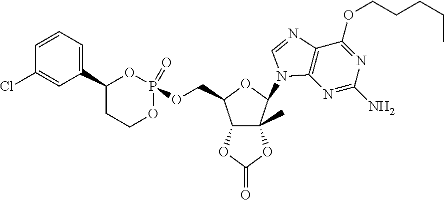 [SMILES: CCCCCOc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 150 was prepared in a similar fashion as described in Example 13 starting from 6-O-pentyl-2′-β-C-methylguanosine instead of 6-O-methyl-2′-β-C-methylguanosine; mp 80-82° C.; MH⁺=624.6.

### Example 42

### 2(R)-(2-Amino-6-isopropyloxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 151)

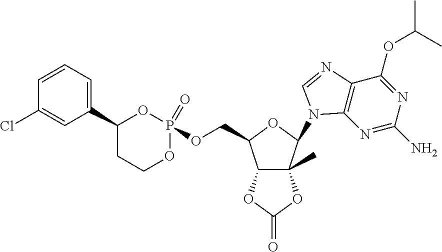 [SMILES: CC(C)Oc1nc(N)nc2c1ncn2[C@@H]1O[C@H](CO[P@@]2(=O)OCC[C@@H](c3cccc(Cl)c3)O2)[C@H]2OC(=O)O[C@]21C]

Compound 151 was prepared in a similar fashion as described in Example 13 starting from 6-O-isopropyl-2′-β-C-methylguanosine instead of 6-O-methyl-2′-β-C-methylguanosine; mp 106-110° C.; MH⁺=596.6.

### Example 43

### 2(R)-(2-Amino-6-ethoxy-9(H)-purinyl)-4(R)-(4(S)-(2-bromophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 152) and 2(R)-(2-amino-6-ethoxy-9(H)-purinyl)-4(R)-(4(R)-(2-bromophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 153)

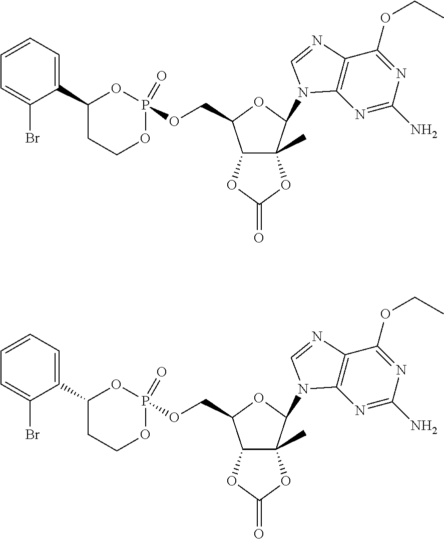 [SMILES: CCOc1nc(N)nc2c1ncn2[C@@H]1O[C@H](CO[P@@]2(=O)OCC[C@@H](c3ccccc3Br)O2)[C@H]2OC(=O)O[C@]21C ; CCOc1nc(N)nc2c1ncn2[C@@H]1O[C@H](CO[P@]2(=O)OCC[C@H](c3ccccc3Br)O2)[C@H]2OC(=O)O[C@]21C]

Compounds 152 and 153 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2-bromophenyl)-2-oxo-1,3,2-dioxaphosphorinane; mp 115-117° C.; MH⁺=628.4.

### Example 44

### 2(R)-(2-Amino-6-ethoxy-9(H)-purinyl)-4(R)-(4(S)-(3-bromo-4-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 154) and 2(R)-(2-amino-6-ethoxy-9(H)-purinyl)-4(R)-(4(R)-(3-bromo-4-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 155)

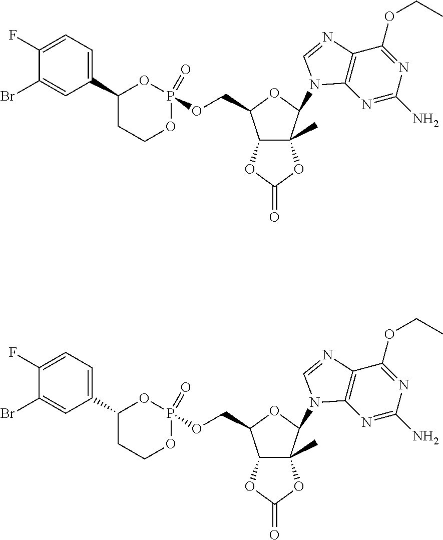 [SMILES: CCOc1nc(N)nc2c1ncn2[C@@H]1O[C@H](CO[P@@]2(=O)OCC[C@@H](c3ccc(F)c(Br)c3)O2)[C@H]2OC(=O)O[C@]21C ; CCOc1nc(N)nc2c1ncn2[C@@H]1O[C@H](CO[P@]2(=O)OCC[C@H](c3ccc(F)c(Br)c3)O2)[C@H]2OC(=O)O[C@]21C]

Compounds 154 and 155 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(3-bromo-4-fluorophenyl)-2-oxo-1,3,2-dioxaphosphorinane; mp 122-124° C.; MH⁺=646.4.

### Example 45

### 2(R)-(2-Amino-6-ethoxy-9(H)-purinyl)-4(R)-(4(S)-(3-trifluoromethylphenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 156) and 2(R)-(2-amino-6-ethoxy-9 (H)-purinyl)-4(R)-(4(R)-(3-trifluoromethylphenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 157)

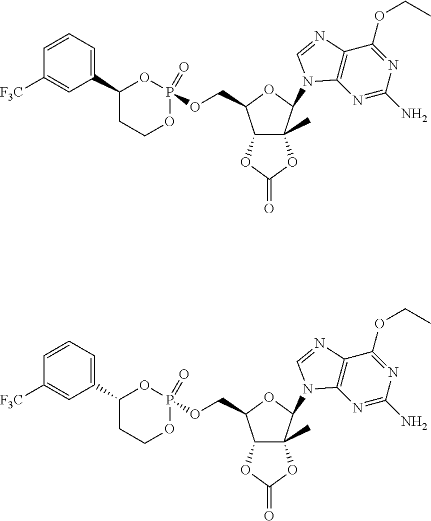 [SMILES: CCOc1nc(N)nc2c1ncn2[C@@H]1O[C@H](CO[P@@]2(=O)OCC[C@@H](c3cccc(C(F)(F)F)c3)O2)[C@H]2OC(=O)O[C@]21C ; CCOc1nc(N)nc2c1ncn2[C@@H]1O[C@H](CO[P@]2(=O)OCC[C@H](c3cccc(C(F)(F)F)c3)O2)[C@H]2OC(=O)O[C@]21C]

Compounds 156 and 157 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(3-trifluoromethylphenyl)-2-oxo-1,3,2-dioxaphosphorinane; mp 94-96° C.; MH⁺=616.4.

### Example 46

### 2(R)-(2-Amino-6-ethoxy-9(H)-purinyl)-4(R)-(4(S)-(4-chloro-2-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 158) and 2(R)-(2-amino-6-ethoxy-9 (H)-purinyl)-4(R)-(4(R)-(4-chloro-2-fluoro phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 159)

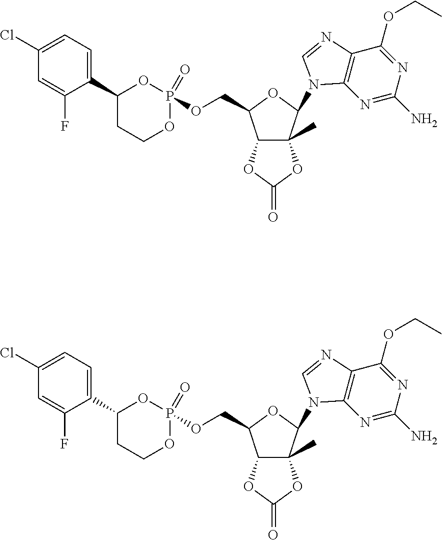 [SMILES: CCOc1nc(N)nc2c1ncn2[C@@H]1O[C@H](CO[P@@]2(=O)OCC[C@@H](c3ccc(Cl)cc3F)O2)[C@H]2OC(=O)O[C@]21C ; CCOc1nc(N)nc2c1ncn2[C@@H]1O[C@H](CO[P@]2(=O)OCC[C@H](c3ccc(Cl)cc3F)O2)[C@H]2OC(=O)O[C@]21C]

Compounds 158 and 159 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(4-chloro-2-fluorophenyl)-2-oxo-1,3,2-dioxaphosphorinane; mp 110-112° C.; MH⁺=600.6.

### Example 47

### 2(R)-(2-Amino-6-ethoxy-9(H)-purinyl)-4(R)-(4(S)-(3-chloro-4-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 160) and 2(R)-(2-amino-6-ethoxy-9(H)-purinyl)-4(R)-(4(R)-(3-chloro-4-fluoro phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 161)

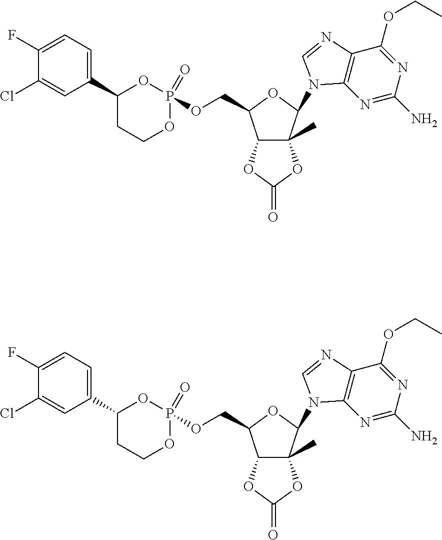 [SMILES: CCOc1nc(N)nc2c1ncn2[C@@H]1O[C@H](CO[P@@]2(=O)OCC[C@@H](c3ccc(F)c(Cl)c3)O2)[C@H]2OC(=O)O[C@]21C ; CCOc1nc(N)nc2c1ncn2[C@@H]1O[C@H](CO[P@]2(=O)OCC[C@H](c3ccc(F)c(Cl)c3)O2)[C@H]2OC(=O)O[C@]21C]

Compounds 160 and 161 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(3-chloro-4-fluorophenyl)-2-oxo-1,3,2-dioxaphosphorinane; mp 107-109° C.; MH⁺=600.4.

### Example 48

### 2(R)-(2-Amino-6-ethoxy-9(H)-purinyl)-4(R)-(4(S)-(2,5-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 162) and 2(R)-(2-amino-6-ethoxy-9(H)-purinyl)-4(R)-(4(R)-(2,5-difluorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 163)

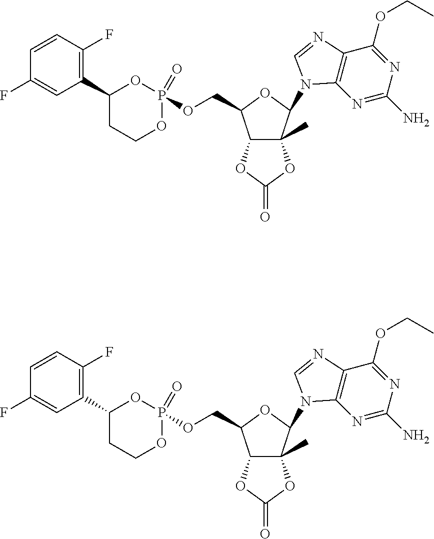 [SMILES: CCOc1nc(N)nc2c1ncn2[C@@H]1O[C@H](CO[P@@]2(=O)OCC[C@@H](c3cc(F)ccc3F)O2)[C@H]2OC(=O)O[C@]21C ; CCOc1nc(N)nc2c1ncn2[C@@H]1O[C@H](CO[P@]2(=O)OCC[C@H](c3cc(F)ccc3F)O2)[C@H]2OC(=O)O[C@]21C]

Compounds 162 and 163 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2,5-difluorophenyl)-2-oxo-1,3,2-dioxaphosphorinane; mp 98-100° C.; MH⁺=584.4.

### Example 49

### 2(R)-(2-Amino-6-ethoxy-9(H)-purinyl)-4(R)-(4(S)-(2-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 164) and 2(R)-(2-amino-6-ethoxy-9(H)-purinyl)-4(R)-(4(R)-(2-chlorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 165)

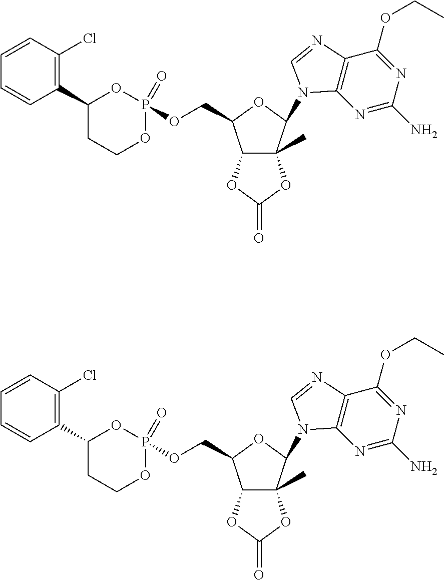 [SMILES: CCOc1nc(N)nc2c1ncn2[C@@H]1O[C@H](CO[P@@]2(=O)OCC[C@@H](c3ccccc3Cl)O2)[C@H]2OC(=O)O[C@]21C ; CCOc1nc(N)nc2c1ncn2[C@@H]1O[C@H](CO[P@]2(=O)OCC[C@H](c3ccccc3Cl)O2)[C@H]2OC(=O)O[C@]21C]

Compounds 164 and 165 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2-chlorophenyl)-2-oxo-1,3,2-dioxaphosphorinane; mp 110-112° C.; MH⁺=582.6.

### Example 50

### 2(R)-(2-Amino-6-ethoxy-9(H)-purinyl)-4(R)-(4(S)-(2,4-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 166) and 2(R)-(2-amino-6-ethoxy-9(H)-purinyl)-4(R)-(4(R)-(2,4-difluorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 167)

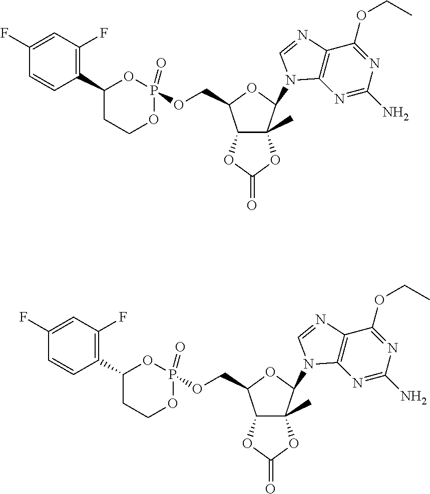 [SMILES: CCOc1nc(N)nc2c1ncn2[C@@H]1O[C@H](CO[P@@]2(=O)OCC[C@@H](c3ccc(F)cc3F)O2)[C@H]2OC(=O)O[C@]21C ; CCOc1nc(N)nc2c1ncn2[C@@H]1O[C@H](CO[P@]2(=O)OCC[C@H](c3ccc(F)cc3F)O2)[C@H]2OC(=O)O[C@]21C]

Compounds 166 and 167 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2,4-difluorophenyl)-2-oxo-1,3,2-dioxaphosphorinane; mp 100-102° C.; MH⁺=584.4.

### Example 51

### 2(R)-(2-Amino-6-ethoxy-9(H)-purinyl)-4(R)-(4(S)-(5-bromo-2-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 168) and 2(R)-(2-amino-6-ethoxy-9(H)-purinyl)-4(R)-(4(R)-(5-bromo-2-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 169)

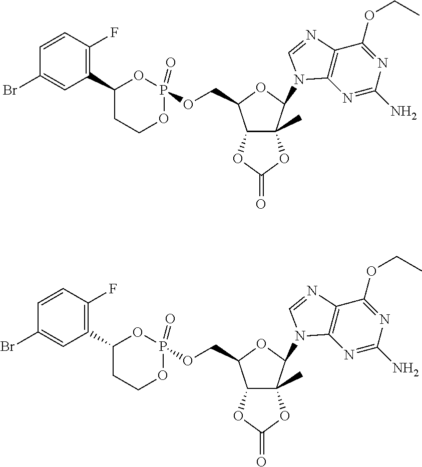 [SMILES: CCOc1nc(N)nc2c1ncn2[C@@H]1O[C@H](CO[P@@]2(=O)OCC[C@@H](c3cc(Br)ccc3F)O2)[C@H]2OC(=O)O[C@]21C ; CCOc1nc(N)nc2c1ncn2[C@@H]1O[C@H](CO[P@]2(=O)OCC[C@H](c3cc(Br)ccc3F)O2)[C@H]2OC(=O)O[C@]21C]

Compounds 168 and 169 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(5-bromo-2-fluorophenyl)-2-oxo-1,3,2-dioxaphosphorinane; mp 107-109° C.; MH⁺=644.4.

### Example 52

### 2(R)-(2-Amino-6-ethoxy-9(H)-purinyl)-4(R)-(4(S)-(2-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 170) and 2(R)-(2-amino-6-ethoxy-9(H)-purinyl)-4(R)-(4(R)-(2-fluoro phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 171)

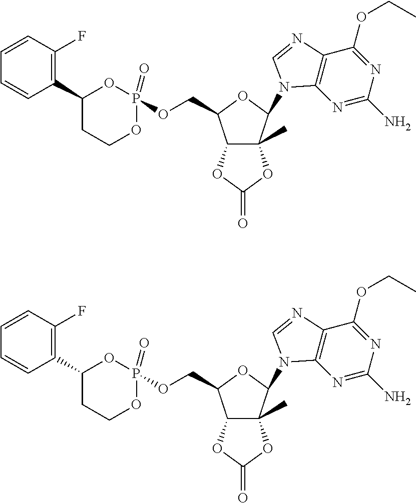 [SMILES: CCOc1nc(N)nc2c1ncn2[C@@H]1O[C@H](CO[P@@]2(=O)OCC[C@@H](c3ccccc3F)O2)[C@H]2OC(=O)O[C@]21C ; CCOc1nc(N)nc2c1ncn2[C@@H]1O[C@H](CO[P@]2(=O)OCC[C@H](c3ccccc3F)O2)[C@H]2OC(=O)O[C@]21C]

Compounds 170 and 171 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2-fluorophenyl)-2-oxo-1,3,2-dioxaphosphorinane; mp 94-96° C.; MH⁺=566.4.

### Example 53

### 2(R)-(2-Amino-6-ethoxy-9(H)-purinyl)-4(R)-(4(S)-(2-chloro-4-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 172) and 2(R)-(2-amino-6-ethoxy-9(H)-purinyl)-4(R)-(4(R)-(2-chloro-4-fluoro phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 173)

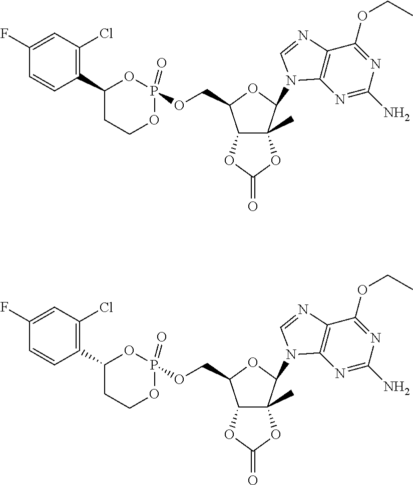 [SMILES: CCOc1nc(N)nc2c1ncn2[C@@H]1O[C@H](CO[P@@]2(=O)OCC[C@@H](c3ccc(F)cc3Cl)O2)[C@H]2OC(=O)O[C@]21C ; CCOc1nc(N)nc2c1ncn2[C@@H]1O[C@H](CO[P@]2(=O)OCC[C@H](c3ccc(F)cc3Cl)O2)[C@H]2OC(=O)O[C@]21C]

Compounds 172 and 173 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2-chloro-4-fluorophenyl)-2-oxo-1,3,2-dioxaphosphorinane; mp 115-117° C.; MH⁺=600.6.

### Example 54

### 2(R)-(2-Amino-6-ethoxy-9(H)-purinyl)-4(R)-(4(S)-(4-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 174) and 2(R)-(2-amino-6-ethoxy-9(H)-purinyl)-4(R)-(4(R)-(4-chlorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 175)

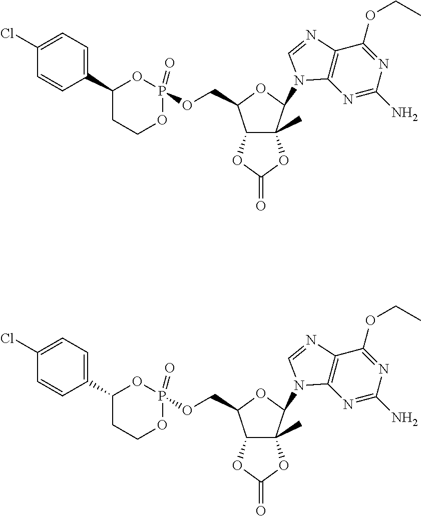 [SMILES: CCOc1nc(N)nc2c1ncn2[C@@H]1O[C@H](CO[P@@]2(=O)OCC[C@@H](c3ccc(Cl)cc3)O2)[C@H]2OC(=O)O[C@]21C ; CCOc1nc(N)nc2c1ncn2[C@@H]1O[C@H](CO[P@]2(=O)OCC[C@H](c3ccc(Cl)cc3)O2)[C@H]2OC(=O)O[C@]21C]

Compounds 174 and 175 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(4-chlorophenyl)-2-oxo-1,3,2-dioxaphosphorinane; mp 111-113° C.; MH⁺=582.6.

### Example 55

### 2(R)-(2-Amino-6-ethoxy-9(H)-purinyl)-4(R)-(4(S)-(2,3-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 176) and 2(R)-(2-amino-6-ethoxy-9(H)-purinyl)-4(R)-(4(R)-(2,3-difluorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 177)

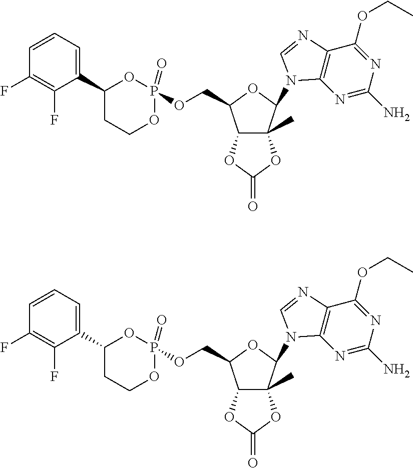 [SMILES: CCOc1nc(N)nc2c1ncn2[C@@H]1O[C@H](CO[P@@]2(=O)OCC[C@@H](c3cccc(F)c3F)O2)[C@H]2OC(=O)O[C@]21C ; CCOc1nc(N)nc2c1ncn2[C@@H]1O[C@H](CO[P@]2(=O)OCC[C@H](c3cccc(F)c3F)O2)[C@H]2OC(=O)O[C@]21C]

Compounds 176 and 177 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2,3-difluorophenyl)-2-oxo-1,3,2-dioxaphosphorinane; mp 82-84° C.; MH⁺=584.4.

### Example 56

### 2(R)-(2-Amino-6-ethoxy-9(H)-purinyl)-4(R)-(4(S)-(2-fluoro-5-methoxyphenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 178) and 2(R)-(2-amino-6-ethoxy-9(H)-purinyl)-4(R)-(4(R)-(2-fluoro-5-methoxyphenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 179)

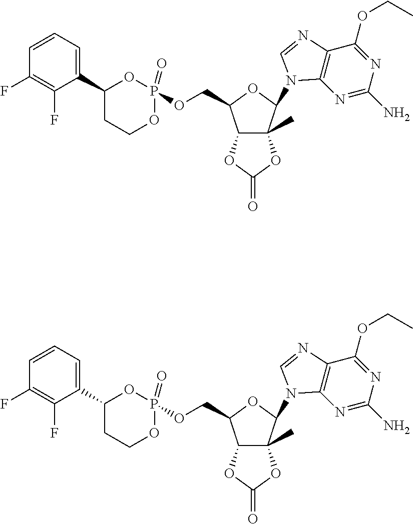 [SMILES: CCOc1nc(N)nc2c1ncn2[C@@H]1O[C@H](CO[P@@]2(=O)OCC[C@@H](c3cccc(F)c3F)O2)[C@H]2OC(=O)O[C@]21C ; CCOc1nc(N)nc2c1ncn2[C@@H]1O[C@H](CO[P@]2(=O)OCC[C@H](c3cccc(F)c3F)O2)[C@H]2OC(=O)O[C@]21C]

Compounds 178 and 179 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2-fluoro-5-methoxyphenyl)-2-oxo-1,3,2-dioxaphosphorinane; mp 94-96° C.; MH⁺=596.6.

### Example 57

### 2(R)-(2-Amino-6-ethoxy-9(H)-purinyl)-4(R)-(4(S)-(2-trifluoromethylphenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 180) and 2(R)-(2-amino-6-ethoxy-9 (H)-purinyl)-4(R)-(4(R)-(2-trifluoromethylphenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 181)

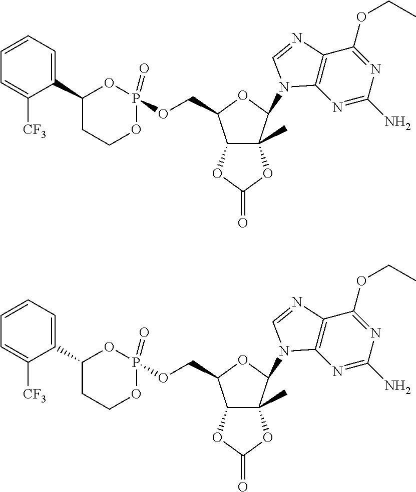 [SMILES: CCOc1nc(N)nc2c1ncn2[C@@H]1O[C@H](CO[P@@]2(=O)OCC[C@@H](c3ccccc3C(F)(F)F)O2)[C@H]2OC(=O)O[C@]21C ; CCOc1nc(N)nc2c1ncn2[C@@H]1O[C@H](CO[P@]2(=O)OCC[C@H](c3ccccc3C(F)(F)F)O2)[C@H]2OC(=O)O[C@]21C]

Compounds 180 and 181 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2-trifluoromethylphenyl)-2-oxo-1,3,2-dioxaphosphorinane; mp 112-114° C.; MH⁺=616.4.

### Example 58

### 2(R)-(2-Amino-6-ethoxy-9(H)-purinyl)-4(R)-(4(S)-phenyl-2-oxo-1,3,2-dioxaphosphorinan-2(R)-yloxymethyl)-1(R)-methyl-7-oxo-3,6,8-trioxa-cis-bicyclo[3.3.0]octane (Compound 182)

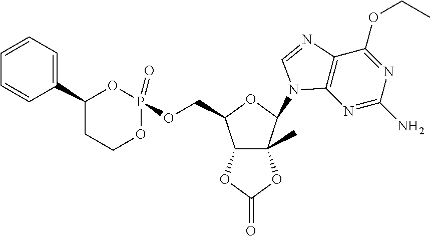 [SMILES: CCOc1nc(N)nc2c1ncn2[C@@H]1O[C@H](CO[P@@]2(=O)OCC[C@@H](c3ccccc3)O2)[C@H]2OC(=O)O[C@]21C]

Compound 182 was prepared in a similar fashion as described in Example 14 by replacing 2(S)-(4-nitrophenoxy)-4(S)-(3-chlorophenyl)-2-oxo-1,3,2-dioxaphosphorinane with 2(S)-(4-nitrophenoxy)-4(S)-phenyl-2-oxo-1,3,2-dioxaphosphorinane; mp 105-107° C.; MH⁺=548.4.

### Example 59

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-acetyloxy-tetrahydrofuran (Compound 183)

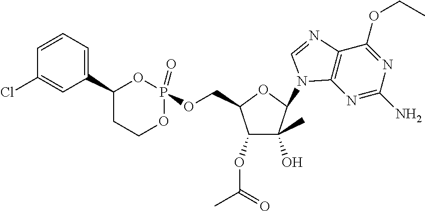 [SMILES: CCOc1nc(N)nc2c1ncn2[C@@H]1O[C@H](CO[P@@]2(=O)OCC[C@@H](c3cccc(Cl)c3)O2)[C@@H](OC(C)=O)[C@@]1(C)O]

Compound 183 was prepared according to synthetic strategy described in Scheme I from 6-O-ethyl-2′-β-C-methylguanosine:

2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-dihydroxy-3(R)-methyltetrahydrofuran: To a solution of 6-O-ethyl-2′-β-C-methylguanosine (0.31 g, 0.95 mmol) in DMF (9 mL) at 0° C. was added dropwise a 1.0 M THF solution of t-BuMgCl (2.4 mL, 2.4 mmol) and the reaction mixture was stirred for 20 min before addition of 2(S)-(4-nitrophenoxy)-4(S)-(3-chlorophenyl)-2-oxo-1,3,2-dioxaphosphorinane (0.42, 1.1 mmol). The reaction was allowed to stir over night and was quenched with saturated aqueous KH₂PO₄ solution (15 mL). Standard work-up followed by chromatography afforded the intermediate as a brown powder (0.14 g, 47% based on recovered starting material); mp 58-60° C.; =556.3.

Compound 183: To a solution of above intermediate (0.12 g, 0.22 mmol) in pyridine (2.2 mL) at room temperature was added acetic anhydride (22 μL, 0.24 mmol) and DMAP (2.6 mg, 0.02 mmol). The reaction was stirred till completion by TLC and standard work-up followed by chromatography provided compound 183 as a white solid (0.12 g, 92%); mp 118-121° C.; MH⁺=598.6.

### Example 60

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-L-valinyl-tetrahydrofuran (Compound 184)

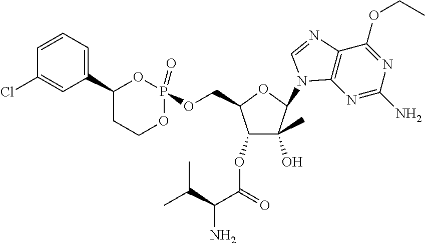 [SMILES: CCOc1nc(N)nc2c1ncn2[C@@H]1O[C@H](CO[P@@]2(=O)OCC[C@@H](c3cccc(Cl)c3)O2)[C@@H](OC(=O)[C@@H](N)C(C)C)[C@@]1(C)O]

Compound 184 was prepared in a similar fashion as described in Example 59 by replacing acetic anhydride with activated L-valine. Compound 184 was isolated as an off-white trifluoroacetate salt; mp 128-130° C.; MH⁺=655.6.

### Example 61

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-isobutyryloxy-tetrahydrofuran (Compound 185)

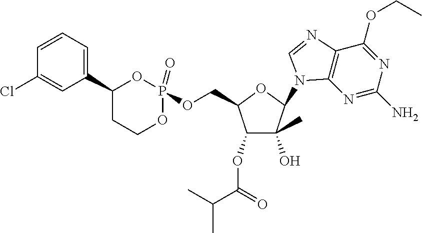 [SMILES: CCOc1nc(N)nc2c1ncn2[C@@H]1O[C@H](CO[P@@]2(=O)OCC[C@@H](c3cccc(Cl)c3)O2)[C@@H](OC(=O)C(C)C)[C@@]1(C)O]

Compound 185 was prepared in a similar fashion as described in Example 59 by replacing acetic anhydride with isobutyric anhydride. Compound 184 was isolated as an off-white trifluoroacetate salt; mp 162-165° C.; MH⁺=626.6.

### Example 62

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-cyclopentylcarboxy-tetrahydrofuran (Compound 186)

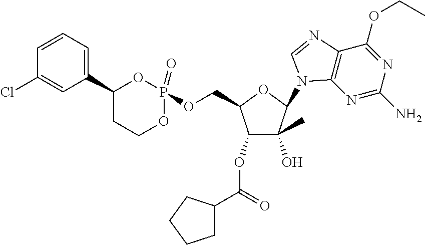 [SMILES: CCOc1nc(N)nc2c1ncn2[C@@H]1O[C@H](CO[P@@]2(=O)OCC[C@@H](c3cccc(Cl)c3)O2)[C@@H](OC(=O)C2CCCC2)[C@@]1(C)O]

Compound 186 was prepared in a similar fashion as described in Example 59 by replacing acetic anhydride with activated cyclopentylcarboxylic acid as a white solid; mp 106-111° C.; MH⁺=652.6.

### Example 63

### 2(R)-(2-Amino-6-ethoxy-9(H)-purinyl)-4(R)-(4(S)-(2,3-dichlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 187) and 2(R)-(2-amino-6-ethoxy-9(H)-purinyl)-4(R)-(4(R)-(2,3-dichlorophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 188)

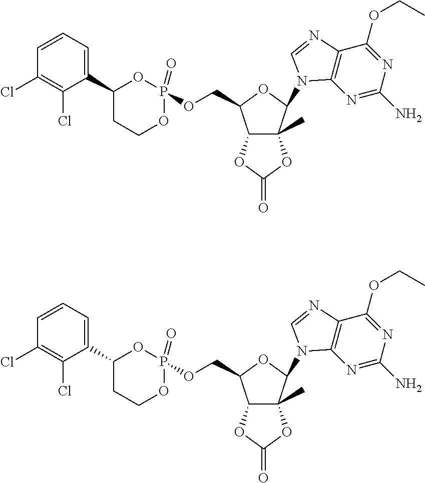 [SMILES: CCOc1nc(N)nc2c1ncn2[C@@H]1O[C@H](CO[P@@]2(=O)OCC[C@@H](c3cccc(Cl)c3Cl)O2)[C@H]2OC(=O)O[C@]21C ; CCOc1nc(N)nc2c1ncn2[C@@H]1O[C@H](CO[P@]2(=O)OCC[C@H](c3cccc(Cl)c3Cl)O2)[C@H]2OC(=O)O[C@]21C]

Compounds 187 and 188 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(2,3-dichlorophenyl)-2-oxo-1,3,2-dioxaphosphorinane; mp 75-77° C.; MH⁺=616.4.

### Example 64

### 2(R)-(2-Amino-6-ethoxy-9(H)-purinyl)-4(R)-(4(S)-(3-chloro-5-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 189) and 2(R)-(2-amino-6-ethoxy-9 (H)-purinyl)-4(R)-(4(R)-(3-chloro-5-fluoro phenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 190)

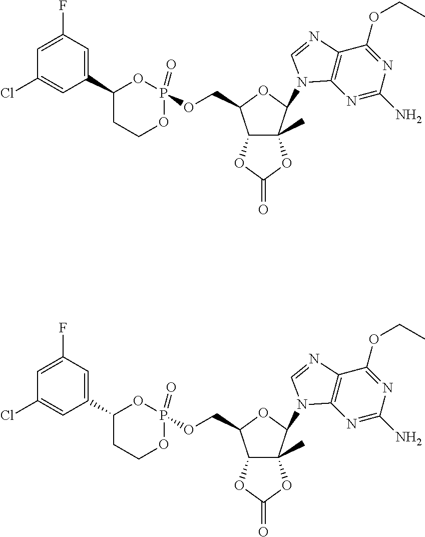 [SMILES: CCOc1nc(N)nc2c1ncn2[C@@H]1O[C@H](CO[P@@]2(=O)OCC[C@@H](c3cc(F)cc(Cl)c3)O2)[C@H]2OC(=O)O[C@]21C ; CCOc1nc(N)nc2c1ncn2[C@@H]1O[C@H](CO[P@]2(=O)OCC[C@H](c3cc(F)cc(Cl)c3)O2)[C@H]2OC(=O)O[C@]21C]

Compounds 189 and 190 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(3-chloro-5-fluorophenyl)-2-oxo-1,3,2-dioxaphosphorinane; mp 106-108° C.; MH⁺=600.6.

### Example 65

### 2(R)-(2-Amino-6-ethoxy-9(H)-purinyl)-4(R)-(4(S)-(3,5-dibromophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 191) and 2(R)-(2-amino-6-ethoxy-9(H)-purinyl)-4(R)-(4(R)-(3,5-dibromophenyl)-2-oxo-1,3,2-dioxaphosphorinan-2(S)-yloxymethyl)-1(R)-methyl-7-oxo-3,6,8-trioxa-cis-bicyclo[3.3.0]octane (Compound 192)

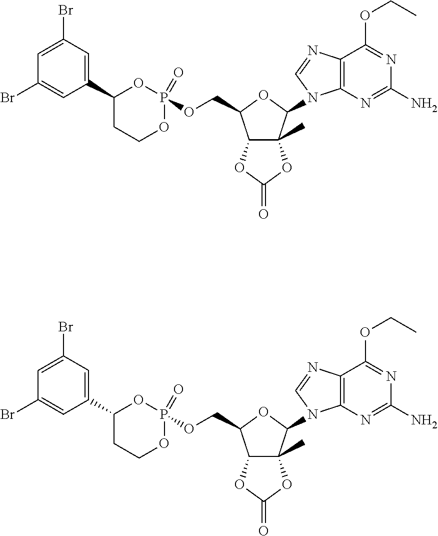 [SMILES: CCOc1nc(N)nc2c1ncn2[C@@H]1O[C@H](CO[P@@]2(=O)OCC[C@@H](c3cc(Br)cc(Br)c3)O2)[C@H]2OC(=O)O[C@]21C ; CCOc1nc(N)nc2c1ncn2[C@@H]1O[C@H](CO[P@]2(=O)OCC[C@H](c3cc(Br)cc(Br)c3)O2)[C@H]2OC(=O)O[C@]21C]

Compounds 191 and 192 were prepared as a 1:1 mixture in a similar fashion as described in Example 14 by replacing 2(S)-(4-nitrophenoxy)-4(S)-(3-chlorophenyl)-2-oxo-1,3,2-dioxaphosphorinane with racemic 2-trans-(4-nitrophenoxy)-4-(3,5-dibromophenyl)-2-oxo-1,3,2-dioxaphosphorinane; mp 120-122° C.; MH⁺=706.4.

### Example 66

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-diacetyloxy-3(R)-methyltetrahydrofuran (Compound 193)

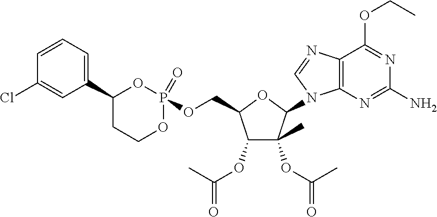 [SMILES: CCOc1nc(N)nc2c1ncn2[C@@H]1O[C@H](CO[P@@]2(=O)OCC[C@@H](c3cccc(Cl)c3)O2)[C@@H](OC(C)=O)[C@@]1(C)OC(C)=O]

Compound 193 can be prepared in the same fashion described in Example 59 from 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-dihydroxy-3(R)-methyltetrahydrofuran by using excess amount of acetic anhydride.

### Example 67

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-dipropionyloxy-3(R)-methyltetrahydrofuran (Compound 194)

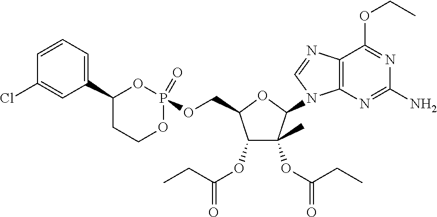 [SMILES: CCOc1nc(N)nc2c1ncn2[C@@H]1O[C@H](CO[P@@]2(=O)OCC[C@@H](c3cccc(Cl)c3)O2)[C@@H](OC(=O)CC)[C@@]1(C)OC(=O)CC]

Compound 194 can be prepared in the same fashion described in Example 59 from 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-dihydroxy-3(R)-methyltetrahydrofuran by using excess amount of propionic anhydride.

### Example 68

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-methoxy-4(R)-propionyloxy-3(R)-methyltetrahydrofuran (Compound 195)

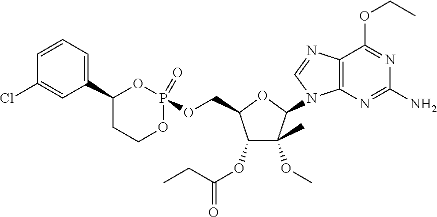 [SMILES: CCOc1nc(N)nc2c1ncn2[C@@H]1O[C@H](CO[P@@]2(=O)OCC[C@@H](c3cccc(Cl)c3)O2)[C@@H](OC(=O)CC)[C@@]1(C)OC]

Compound 195 can be prepared according to the general procedure of Scheme I.

### Example 69

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-methoxy-4(R)-acetyloxy-3(R)-methyltetrahydrofuran (Compound 196)

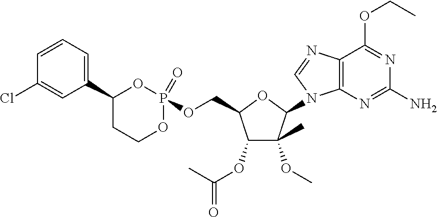 [SMILES: CCOc1nc(N)nc2c1ncn2[C@@H]1O[C@H](CO[P@@]2(=O)OCC[C@@H](c3cccc(Cl)c3)O2)[C@@H](OC(C)=O)[C@@]1(C)OC]

Compound 196 can be prepared according to the general procedure of Scheme I.

### Example 70

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-fluoro-4(R)-acetyloxy-3(R)-methyltetrahydrofuran (Compound 197)

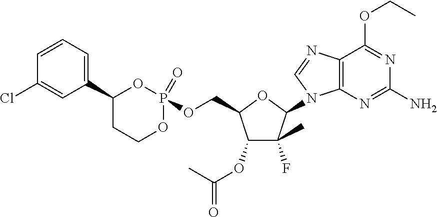 [SMILES: CCOc1nc(N)nc2c1ncn2[C@@H]1O[C@H](CO[P@@]2(=O)OCC[C@@H](c3cccc(Cl)c3)O2)[C@@H](OC(C)=O)[C@@]1(C)F]

Compound 197 can be prepared according to the general procedure of Scheme I.

### Example 71

### 2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-fluoro-4(R)-propionyloxy-3(R)-methyltetrahydrofuran (Compound 198)

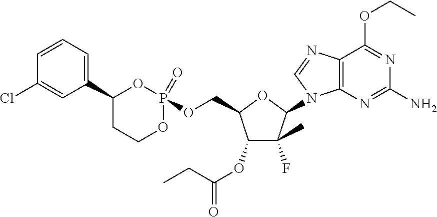 [SMILES: CCOc1nc(N)nc2c1ncn2[C@@H]1O[C@H](CO[P@@]2(=O)OCC[C@@H](c3cccc(Cl)c3)O2)[C@@H](OC(=O)CC)[C@@]1(C)F]

Compound 198 can be prepared according to the general procedure of Scheme I.

### Example 72

### 2(R)-Uracilyl-5(R)-(4(S)-(3-chlorophenyl)-2-oxo-1,3,2-dioxaphosphorinan-2(R)-yloxymethyl)-3(R)-hydroxy-4(R)-propionyloxy-3(R)-methyltetrahydrofuran (Compound 199)

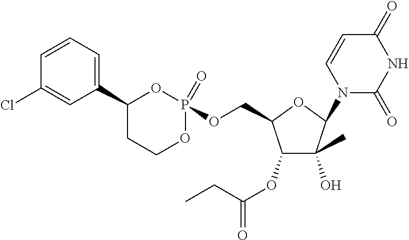 [SMILES: CCC(=O)O[C@@H]1[C@@H](CO[P@@]2(=O)OCC[C@@H](c3cccc(Cl)c3)O2)O[C@@H](n2ccc(=O)[nH]c2=O)[C@]1(C)O]

Compound 199 can be prepared according to the general procedure of Scheme I.

### Example 73

### 2(R)-Uracilyl-5(R)-(4(S)-(3-chloro phenyl)-2-oxo-1,3,2-dioxaphosphorinan-2(R)-yloxymethyl)-3(R)-hydroxy-4(R)-acetyloxy-3(R)-methyltetrahydrofuran (Compound 200)

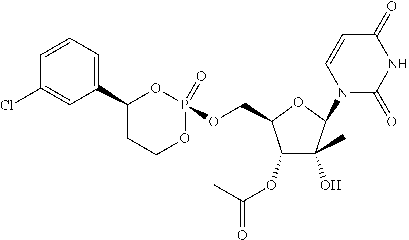 [SMILES: CC(=O)O[C@@H]1[C@@H](CO[P@@]2(=O)OCC[C@@H](c3cccc(Cl)c3)O2)O[C@@H](n2ccc(=O)[nH]c2=O)[C@]1(C)O]

Compound 200 can be prepared according to the general procedure of Scheme I.

### Example 74

### 2(R)-Uracilyl-5(R)-(4(S)-(3-chlorophenyl)-2-oxo-1,3,2-dioxaphosphorinan-2(R)-yloxymethyl)-3(R)-methoxy-4(R)-acetyloxy-3(R)-methyltetrahydrofuran (Compound 201)

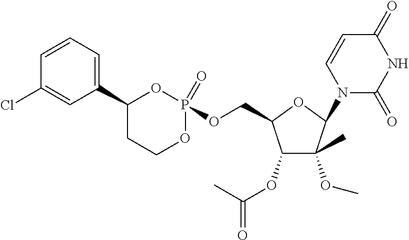 [SMILES: CO[C@]1(C)[C@H](OC(C)=O)[C@@H](CO[P@@]2(=O)OCC[C@@H](c3cccc(Cl)c3)O2)O[C@H]1n1ccc(=O)[nH]c1=O]

Compound 201 can be prepared according to the general procedure of Scheme I.

### Example 75

### 2(R)-Uracilyl-5(R)-(4(S)-(3-chlorophenyl)-2-oxo-1,3,2-dioxaphosphorinan-2(R)-yloxymethyl)-3(R)-methoxy-4(R)-propionyloxy-3(R)-methyltetrahydrofuran (Compound 202)

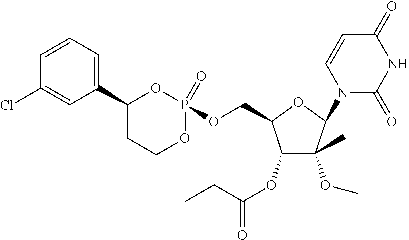 [SMILES: CCC(=O)O[C@@H]1[C@@H](CO[P@@]2(=O)OCC[C@@H](c3cccc(Cl)c3)O2)O[C@@H](n2ccc(=O)[nH]c2=O)[C@]1(C)OC]

Compound 202 can be prepared according to the general procedure of Scheme I.

### Example 76

### 2(R)-Uracilyl-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane (Compound 203)

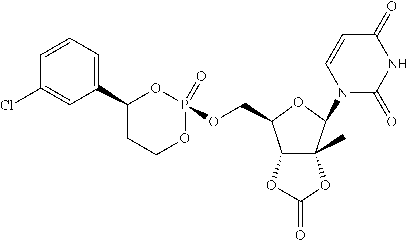 [SMILES: C[C@@]12OC(=O)O[C@@H]1[C@@H](CO[P@@]1(=O)OCC[C@@H](c3cccc(Cl)c3)O1)O[C@H]2n1ccc(=O)[nH]c1=O]

Compound 203 can be prepared according to the general procedure of Scheme I.

### Biological Examples

It will be understood that these examples are exemplary and that the method described herein is not limited solely to these examples.

For the purposes of clarity and brevity, chemical compounds are referred to as synthetic example numbers in the biological examples below.

### Example A

### In Vitro Activation of Prodrug Analogues by Rat Liver Microsomes Quantification by by-Product Capture

The prodrug analogues is tested for activation in rat liver microsomes by means of a prodrug byproduct capture assay.

### Methods:

Prodrugs are tested for activation by liver microsomes isolated from rats induced with dexamethasone to enhance CYP3A4 activity (Human Biologics Inc., Phoenix Ariz.). The study is performed at 2 mg/mL rat liver microsomes, 100 mM KH₂PO₄, 10 mM glutathione, 25 μM or 250 μM compound, and 2 mM NADPH for 0-7.5 min. in an Eppendorf Thermomixer 5436 at 37° C., speed 6. The reactions are initiated by addition of NADPH following a 2-min. preincubation. Reactions are quenched with 60% methanol at 0, 2.5, 5, and 7.5 min. L-Glutamyl-L-(S-(3-oxo-3-(3-chlorophenyl)propyl)cysteinylglycine, a glutathione adduct of the by-product of prodrug activation, 3-chlorophenyl vinyl ketone, is quantified following extraction of the reaction with 1.5 volumes of methanol. The extracted samples are centrifuged at 14,000 rpm in an Eppendorf microfuge and the supernatant analyzed by HPLC for L-glutamyl-L-(S-(3-oxo-3-(3-chlorophenyl)propyl)cysteinylglycine content. Spiked L-glutamyl-L-(S-(3-oxo-3-(3-chlorophenyl)propyl)cysteinylglycine standards (1-30 μM) are prepared in 2 mg/mL microsomes under reaction conditions and then quenched and processed in an identical fashion to unknown samples. For HPLC analysis, the loading mobile phase buffer (Buffer A) consists of a 9:1 ratio (v/v) of 20 mM potassium phosphate, pH 6.2 and acetonitrile. Extract (100 μL) is injected onto a Beckman Ultrasphere ODS column (4.6×250 mM). The column is eluted with a gradient to 60% acetonitrile.

The elution of L-glutamyl-L-(S-(3-oxo-3-(3-chlorophenyl)propyl)cysteinylglycine (retention time 10.4 min.) is monitored at 245 nm. The results are obtained as the activation of compounds in rat liver microsomes, in nmol/mg/min.

Formation of product, L-glutamyl-L-(S-(3-oxo-3-(3-chlorophenyl)propyl) cysteinylglycine indicates activation of a prodrug compound.

### Example B

### In Vitro Activation of Prodrug Analogues by Rat Liver Microsomes. Quantification by LC-MS/MS

Prodrug analogues are tested for activation to NMP in reactions catalyzed by the microsomal fraction of rat liver.

### Methods:

Prodrugs are tested for activation by liver microsomes isolated from rats induced with dexamethasone to enhance CYP3A4 activity (Human Biologics Inc., Phoenix Ariz.). Reactions are conducted in 0.1 M KH₂PO₄, pH 7.4, in the presence of 2 mM NADPH and liver microsomes (1 mg/mL). Reaction mixtures are incubated for 5 min. in an Eppendorf Thermomixer 5436 (37° C., speed 6). Reactions are terminated by the addition of 1.5 volumes of methanol. The resulting extracts are clarified by centrifugation at 14,000 rpm in an Eppendorf microfuge (20 min.). The supernatants (200 μL) are evaporated under vacuum and heat to dryness. The dried residue is reconstituted with 200 μL of water and the mixture is centrifuged for 10 min at 14,000 rpm. A mixture of 35 μL aliquot of supernatant and 35 μL of mobile phase A (20 mM N—N-dimethylhexylamine and 10 mM propionic acid in 20% methanol) is analyzed by LC-MS/MS (Applied Biosystems, API 4000) equipped with an Agilent 1100 binary pump and a LEAP injector. NMP is detected by using MS/MS mode (M⁻/78.8) and quantified based on comparison to a standard of lamivudine monophosphate.

The results are obtained as the activation of compounds in Rat Liver Microsomes, in nmol/mg/min.

### Example C

### In Vitro Activation in Human Liver Microsomes. Quantification by by-Product Capture

The prodrug analogues are tested for activation in human liver microsomes.

### Methods:

Human liver microsomes are purchased from In Vitro Technologies (IVT1032). The study is performed at 2 mg/mL human liver microsomes, 100 mM KH₂PO₄, 10 mM glutathione, 25 μM or 250 μM compound, and 2 mM NADPH for 0-7.5 min. in an Eppendorf Thermomixer 5436 at 37° C., speed 6. The reactions are initiated by addition of NADPH following a 2-min. preincubation. Reactions are quenched with 60% methanol at 0, 2.5, 5, and 7.5 min. L-Glutamyl-L-(S-(3-oxo-3-(3-chlorophenyl)propyl)cysteinylglycine, a glutathione adduct of the by-product of prodrug activation, 3-chlorophenyl vinyl ketone, is quantified following extraction of the reaction with 1.5 volumes of methanol. The extracted samples are centrifuged at 14,000 rpm in an Eppendorf microfuge and the supernatant analyzed by HPLC for L-glutamyl-L-(S-(3-oxo-3-(3-chlorophenyl)propyl)cysteinylglycine content. Spiked L-glutamyl-L-(S-(3-oxo-3-(3-chlorophenyl)propyl)cysteinylglycine standards (1-30 μM) are prepared in 2 mg/mL microsomes under reaction conditions and then quenched and processed in an identical fashion to unknown samples. For HPLC analysis, the loading mobile phase buffer (Buffer A) consists of a 9:1 ratio (v/v) of 20 mM potassium phosphate, pH 6.2 and acetonitrile. Extract (100 μL) is injected onto a Beckman Ultrasphere ODS column (4.6×250 mM). The column is eluted with a gradient to 60% acetonitrile. The elution of L-glutamyl-L-(S-(3-oxo-3-(3-chlorophenyl)propyl)cysteinylglycine (retention time 10.4 min.) is monitored at 245 nm.

Formation of product, L-glutamyl-L-(S-(3-oxo-3-(3-chlorophenyl)propyl) cysteinylglycine indicates the prodrugs are activated in vitro in human liver microsomes.

### Example D

### In Vitro Activation of Prodrug Analogues by Human Liver Microsomes. Quantification by LC-MS/MS

Prodrug analogues are tested for activation to NMP in reactions catalyzed by the microsomal fraction of human liver.

### Methods:

Prodrugs are tested for activation by human liver microsomes purchased from In Vitro Technologies (IVT1032) Reactions are conducted in 0.1 M KH₂PO₄, pH 7.4, in the presence of 2 mM NADPH and liver microsomes (1 mg/mL). Reaction mixtures are incubated for 5 min. in an Eppendorf Thermomixer 5436 (37° C., speed 6). Reactions are terminated by the addition of 1.5 volumes of methanol. The resulting extracts are clarified by centrifugation at 14,000 rpm in an Eppendorf microfuge (20 min.). The supernatants (200 μL) are evaporated under vacuum and heated to dryness. The dried residue is reconstituted with 200 μL of water and the mixture is centrifuged for 10 min at 14,000 rpm. A mixture of 35 μL aliquot of supernatant and 35 μL of mobile phase A (20 mM N—N-dimethylhexylamine and 10 mM propionic acid in 20% methanol) is analyzed by LC-MS/MS (Applied Biosystems, API 4000) equipped with an Agilent 1100 binary pump and a LEAP injector. NMP is detected by using MS/MS mode (M⁻/78.8) and quantified based on comparison to a standard of lamivudine monophosphate. The results are obtained as the activation of compounds in human liver microsomes, in nmol/mg/min.

### Example E

### NTP Accumulation in Hepatocytes Following Incubation with Nucleoside Analogues and their Prodrugs

Nucleoside analogues and their prodrugs are evaluated for their ability to generate NTPs in freshly isolated rat hepatocytes. It is generally accepted that the NTP form of a nucleoside is the active antiviral agent.

### Methods:

Hepatocytes are prepared from fed Sprague-Dawley rats (250-300 g) according to the procedure of Berry and Friend (Berry, M. N. Friend, D. S., J. Cell Biol. 43:506-520 (1969)) as modified by Groen (Groen, A. K. et al., Eur. J. Biochem 122:87-93 (1982)). Hepatocytes (20 mg/mL wet weight, >85% trypan blue viability) are incubated at 37° C. in 2 mL of Krebs-bicarbonate buffer containing 20 mM glucose, and 1 mg/mL BSA for 2 h in the presence of 1-250 μM nucleoside or prodrug (from 25 mM stock solutions in DMSO). Following the incubation, 1600 μL aliquot of the cell suspension is centrifuged and 300 μL of acetonitrile is added to the pellet, vortexed and sonicated until the pellet breaks down. Then 200 μL of water is added to make a 60% acetonitrile solution. After 10 min centrifugation at 14,000 rpm, the resulting supernatant is transferred to a new vial and evaporated to near dryness in a Savant SpeedVac Plus at room temperature. The dried residue is reconstituted with 200 μL of water and the mixture is centrifuged for 10 min at 14,000 rpm. A mixture of 35 μL aliquot of supernatant and 35 μL of mobile phase A (20 mM N—N-dimethylhexylamine and 10 mM propionic acid in 20% methanol) is analyzed by LC-MS/MS (Applied Biosystems, API 4000) equipped with an Agilent 1100 binary pump and a LEAP injector. NTP is detected by using MS/MS mode (M⁻/78.8) and quantified based on comparison to a standard of lamivudine triphosphate.

Following the incubation of 25 μM or 250 μM nucleosides and prodrugs with primary rat hepatocytes, NTP formation is observed over the course of 2 h, and is measured as nmol/g.

### Example F

### HCV-Infected Human Liver Slice Assay

Inhibition of HCV replication in human liver tissue is evaluated using the following assay.

### Methods:

Procurement. Liver from a brain-dead HCV antibody-positive human patient is perfused with ice-cold Viaspan (Dupont Pharmaceutical) preservation solution and received on ice in Viaspan.

Precision-cut liver slices of ˜200-250 μm thickness and 8 cm diameter are prepared and cultured in Waymouth's cell culture medium (Gibco, Inc.) that is supplemented with 10% fetal bovine serum and 10 mL/L Fungi-Bact at 37° C., and gassed with carbogen (95% O₂, 5% CO₂) at 0.75 liters/min. Tissue slices are maintained in culture for 72 h. Cell culture medium containing test compound in solution is changed on a daily basis.

At appropriate times of liver slice incubation, liver slices and medium are collected for analysis of HCV RNA (tissue and medium) and nucleotide levels (NTP). All collected media and tissue slices are maintained in liquid N₂ until analysis.

Medium and tissue samples are analyzed for HCV RNA levels according to published procedures (Bonacini et. al., 1999) which utilize an automated, multicycle, polymerase chain reaction (PCR) based technique. This assay has a lower limit of detection for HCV RNA of 100 viral copies/ml.

### Analysis of Tissue NTPs:

Frozen liver slices are disrupted by using a combination of ultrasound probe sonication, Branson Sonifier 450 (Branson Ultrasonics, Danbury, Conn.) and homogenization using a Dounce conical pestle in 200 μls of 10% (v/v) perchloric acid (PCA). After a 5 min centrifugation at 2,500×g, the supernatants are neutralized using 3 M KOH/3 M KHCO₃ and mixed thoroughly. The neutralized samples are centrifuged at 2,500 g for 5 min and NTP levels are determined by ion exchange phase HPLC (Hewlett Packard 1050) using a Whatman Partisil 5 SAX (5 μm, 4.6×250 mm) column. Samples (50 μL) are injected onto the column in 70% 10 mM ammonium phosphate buffer and 30% 1 M ammonium phosphate buffer, both at pH 3.5 and containing 6% ethanol. Nucleoside triphosphates are eluted from the column using a linear gradient to 80% 1 M ammonium phosphate pH 3.5/6% ethanol buffer, at a flow rate of 1.25 mL/min and detected by UV absorbance (254 nm).

HCV RNA levels present in the liver slice culture media decreased from the levels present in control, untreated slices following incubation with 2′-C-methylguanosine and compound TBD.

Treatment of HCV-infected human liver slices with 2′-C-methylguanosine or compound TBD for 72 h decreases the amount of HCV RNA released into the culture medium from 48-72 h. Treatment with the prodrug, is more effective than treatment with the nucleoside, 2′-C-methylguanosine, at lowering viral RNA production in the culture medium.

### Example G

### Liver Targeting of Nucleoside Analogues and their Prodrugs

The liver specificity of a compound is compared relative to the parent nucleoside, 2′-C-methylguanosine, or 2′-C-methyladenosine, by measuring the generation of NTP in the liver compared to nucleoside in the plasma.

### Methods:

A compound or 2′-C-methylguanosine are administered intraperitoneally to C57BL/6 mice at a dose of 30 mg/kg based on nucleoside equivalents (30 mg/kg 2′-C-methylguanosine and 53.27 mg/kg compound TBD). A compound or 2′-C-methyladenosine is administered intravenously to C57BL/6 mice at dose of about 5.5 mg/kg nucleoside equivalents (5.5 mg/kg 2′-C-methyladenosine and 10 mg/kg compound TBD). Plasma concentrations of 2′-C-methylguanosine, compound 19, 2′-C-methyladenosine, and a compound are determined by HPLC-UV and the liver concentrations of the 5′-triphosphate of 2′-C-methylguanosine and 2′-C-methyladenosine are measured by LC-MS using the standard ion-pairing chromatography method for triphosphate as described in Example E. Conventional SAX HPLC-UV can not differentiate between endogenous GTP and 2′-C-methylguanosine triphosphate. Since an authentic standard of 2′-C-methylguanosine triphosphate is not available, the liver concentrations of the nucleotide are approximated as described in Example E.

Liver targeting of a compound as the triphosphate of 2′-C-methylguanosine and of a compound as the triphosphate of 2′-C-methyladenosine are clearly demonstrated with the prodrugs.

### Example H

### Tissue Distribution Following Oral Administration of Nucleoside Analogues and their Prodrugs

The liver specificity of prodrugs are compared relative to their parent nucleoside analog inhibitors in liver and other organs that could be targets of toxicity.

### Methods:

Nucleoside analogues and their prodrugs are administered at 30 mg/kg (in terms of nucleoside equivalents) to fasted rats by oral gavage. Plasma concentrations of nucleoside and prodrug are determined by HPLC-UV, as described in Example J, and the liver, skeletal muscle, cardiac, kidney, small intestine, and other organ concentrations of the 5′-triphosphate of the nucleoside are measured by LC-MS using the standard ion-pairing chromatography method for triphosphate as described in Example E.

The results demonstrate the liver targeting of the nucleoside analog prodrugs and provide evidence for improved safety of the prodrugs over that of the nucleosides alone. This can occur solely by the liver targeting provided by the prodrug or by additional liver metabolism of nucleoside derived following dephosphorylation of the nucleoside monophosphate. In the latter case, the nucleoside can escape from the liver into the periphery leading to exposure of other tissues to the nucleoside and potential extrahepatic toxicity. The release of nucleoside from the liver can be reduced by metabolism of the nucleoside monophosphate or the nucleoside in the liver cell, e.g. the breakdown of adenosine-based nucleoside monophosphates to inosine via adenylate deaminase and nucleotidase, or the breakdown of adenosine-based nucleoside to inosine and hypoxanthine by adenosine deaminase and purine nucleoside phosphorylase, respectively.

### Example I

### Assessment of the Oral Bioavailability of Nucleoside Analogues and their Prodrugs in Normal Male Rats

The oral bioavailability (OBAV) of the nucleoside analogues and their prodrugs are evaluated in the normal male rat.

### Methods:

The compounds are solubilized in a suitable vehicle for intravenous and oral administration. The OBAV is assessed by calculating the ratio of the AUC values of the liver organ concentration-time profile of NTP following oral and i.v. or i.p. administration of 30 mg/kg (in terms of nucleoside equivalents) of the compound to groups of four rats. Liver organ samples are taken at 20 min and 1, 3, 5, 8, 12, and 24 h following dosing. Liver organ concentrations of NTP are determined as determined by LC-MS/MS (Example E) or HPLC (Example F) analysis.

### Example J

### Susceptibility of Nucleoside Analogues to Metabolism in Rat Liver S9 Fraction or Isolated Hepatocytes

The susceptibility of the purine nucleoside analogues to metabolism is assessed in rat liver S9 fraction or isolated rat hepatocytes.

### Methods:

Purine nucleoside analogues (100 μM) (e.g., 2′-C-methyladenosine) are incubated in rat liver S9 fraction or with isolated rat hepatocytes at 37° C. The reactions are terminated at time points up to 2 h and then deproteinized by extraction with 60% acetonitrile. Following centrifugation, the supernatants are evaporated to dryness and the resulting residues are reconstituted with aqueous mobile phase. These samples are analyzed for potential metabolites by a single HPLC system equipped with a diode array detector. Nucleosides (e.g., 2′-C-methylinosine) and bases (e.g., hypoxanthine) are separated and quantified on a Beckman Ultrasphere C-18 reverse phase column (4.5×250 mm) using a gradient of Buffer A (100 mM potassium phosphate pH 6) and Buffer B (25% v/v methanol) at a flow rate of 1.5 mL/min. The column is developed over 40 min using a nonlinear gradient of 0% Buffer B to 100% Buffer B (% pump Buffer B=100×(time [min]/40)3) and monitored by UV absorbance at 260 nm. Metabolites are identified by coelution with authentic standards and/or UV spectrum matching.

The susceptibility of the purine nucleoside analogues to metabolism is dependent upon the type and location of the structural modification of the congener. The inclusion of certain pharmacophores (such as the 2′-C-methyl group of 2′-C-methyladenosine] leads to increased resistance to metabolism by purine salvage pathway enzymes such as adenosine deaminase and purine nucleoside phosphorylase) as described in Eldrup A B, Allerson C R, Bennett C F, et al. (2004) J. Med. Chem. 47(9):2283-2295, “Structure-activity relationship of purine ribonucleotides for inhibition of hepatitis C virus RNA-dependent RNA polymerase.”

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present application, and is susceptible to modifications in the methods and materials, as well as alterations in the fabrication methods and equipment. Such modifications will become apparent to those skilled in the art from a consideration of this disclosure herein. Consequently, it is not intended that the present application be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A compound selected from the group consisting of the compounds of Formula I, and the compounds of Formula II: 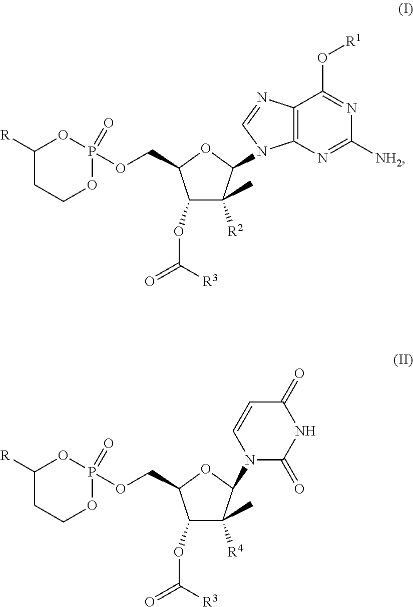
wherein:
R is an optionally substituted phenyl or an optionally substituted pyridyl;
R¹ is selected from the group consisting of hydrogen, a C₁-C₁₀ alkyl, C₁-C₁₀ alkenyl, and a C₁-C₁₀ heteroalkyl;
R² is selected from the group consisting of OR⁵, acyloxy, and F;
R³ is a C₁-C₆ alkyl or a C₁-C₆ heteroalkyl, or R² and R³ can be linked to form a —O— bond;
R⁴ is selected from the group consisting of OR⁵ and acyloxy, or R³ and R⁴ can be linked to form a —O— bond; and
R⁵ is selected from the group consisting of hydrogen, a C₁-C₁₀ alkyl, a C₁-C₁₀ haloalkyl, and a C₁-C₁₀ heteroalkyl, and the stereoisomers and the pharmaceutically acceptable salts thereof.

2. The compound of claim 1 wherein R is selected from the group consisting of:

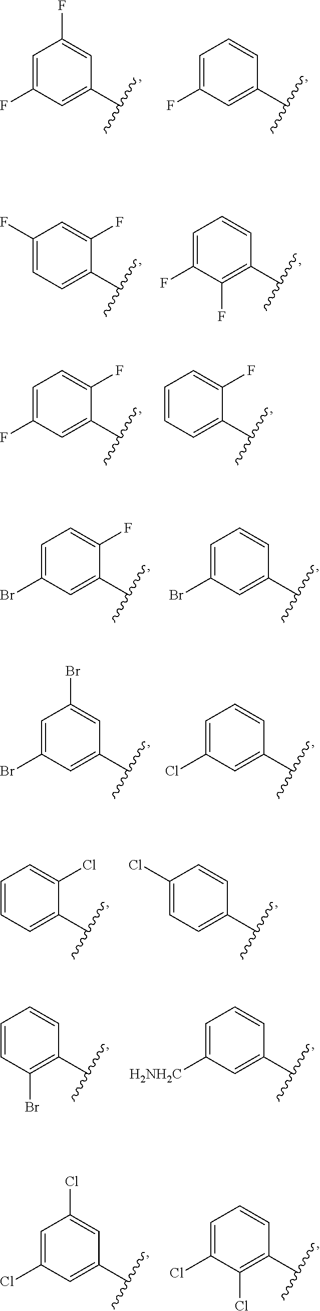

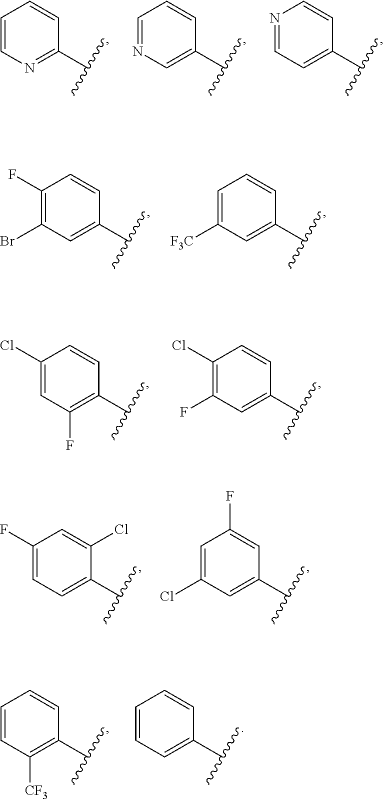

3. The compound of claim 1, wherein R¹ is hydrogen.

4. The compound of claim 1, wherein R¹ is methyl.

5. The compound of claim 1, wherein R¹ is ethyl.

6. The compound of claim 2, wherein R¹ is selected from the group consisting of

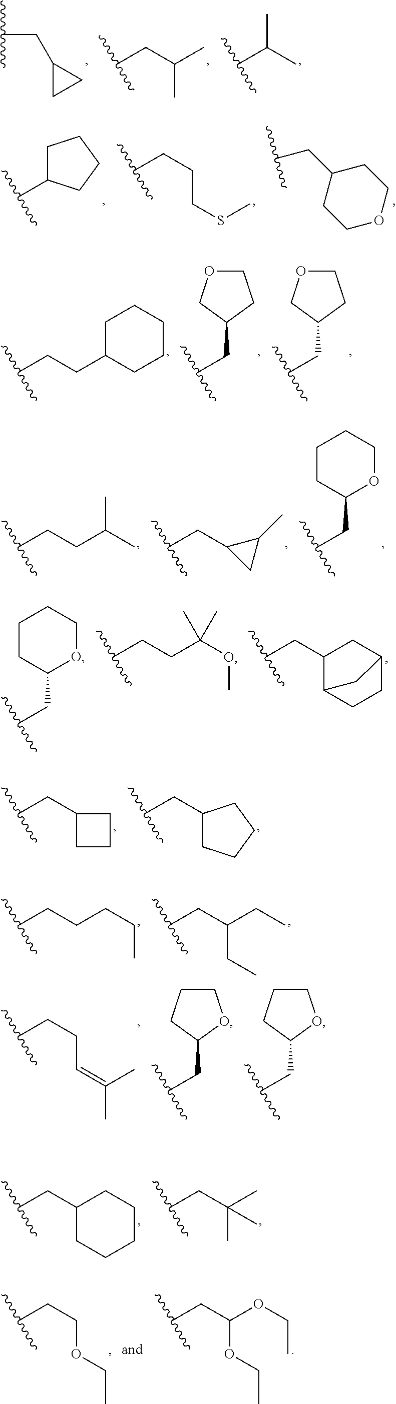

7. The compound of claim 1, wherein R² and R³ are linked to form a —O— bond.

8. The compound of claim 1, wherein R⁵ is hydrogen.

9. The compound of claim 1, wherein R² or R⁴ is fluorine.

10. The compound of claim 1, wherein R² or R⁴ is methyl.

11. The compound of claim 1, wherein R² or R⁴ is selected from the group consisting of

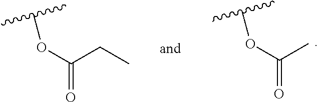

12. The compound of claim 6, wherein R³ is methyl.

13. The compound of claim 6, wherein R³ is isopropyl.

14. The compound of claim 6, wherein R³ is ethyl.

15. The compound of claim 6, wherein R³ is cyclopentyl.

16. The compound of claim 6, wherein R³ is

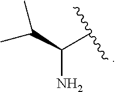 [SMILES: *[C@@H](N)C(C)C]

17. A pharmaceutical composition comprising a therapeutically effective amount of a compound of claim 1, and at least one pharmaceutically acceptable carrier, diluent, or lubricant.

18. A method of treating a viral infection comprising administering to a subject suffering from the viral infection a therapeutically effective amount of a compound of claim 1.

19. (canceled)

20. The method of claim 18, wherein the viral infection is a viral infection of the liver.

21. The method of claim 18, wherein the viral infection is an RNA-dependent RNA viral infection.

22. The method of claim 18, wherein the viral infection is HCV.

23. A method of inhibiting viral replication in a human patient comprising administering to said human patient a therapeutically effective amount of a compound of claim 1.

24. (canceled)

25. The method of claim 23, wherein the viral replication is RNA-dependent RNA viral replication.

26. The method of claim 23, wherein the viral replication is HCV replication.

27. The method of claim 23, wherein said compound of Formula I is used in combination with a therapeutically effective amount of a second agent that is active against HCV.

28. The method of claim 27, wherein said second agent active against HCV is selected from the group consisting of ribavirin, levovirin, viramidine, thymosin alpha-1, interferon-β, an inhibitor of NS3 serine protease, an inhibitor of inosine monophosphate dehydrogenase, interferon-α, pegylated interferon-α, amantadine, a branched ribonucleoside selected from the group consisting of a 1′-C-branched ribonucleoside, a 2′-C-branched ribonucleoside, and a 3′-C-branched ribonucleoside, and combinations thereof.

29. (canceled)

30. (canceled)

31. The method of claim 28, wherein the branched ribonucleoside is selected from the group consisting of 2′-C-methylcytidine, 2′-C-methyluridine, 2′-C-methyladenosine, 2′-C-methylguanosine, and 9-(2-C-methyl-β-D-ribofuranosyl)-2,6-diaminopurine.

32. The compound of claim 1 selected from the group consisting of:
2(R)-(2-Amino-6-hydroxy-9(H)-purinyl)-4(R)-(4(S)-(3,5-difluorophenyl)-2-oxo-1,3,2-dioxaphosphorinan-2-(R)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(3-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-hydroxy-9(H)-purinyl)-4(R)-(4(R)-(3-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(3-bromophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-hydroxy-9(H)-purinyl)-4(R)-(4(R)-(3-bromophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(2-bromophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-hydroxy-9(H)-purinyl)-4(R)-(4(R)-(2-bromophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(3-cyanophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(2-pyridyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-hydroxy-9(H)-purinyl)-4(R)-(4(R)-(2-pyridyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(3-pyridyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-hydroxy-9(H)-purinyl)-4(R)-(4(R)-(3-pyridyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(3,5-dichlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-hydroxy-9(H)-purinyl)-4(R)-(4(R)-(3,5-dichlorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-hydroxy-9(H)-purinyl)-4(R)-(4(S)-(4-pyridyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-hydroxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-(2-methyl-1-oxopropoxy)-tetrahydrofuran;
2(R)-(2-Amino-6-hydroxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-L-valinyl-tetrahydrofuran;
2(R)-(2-Amino-6-methoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-cyclopropylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2-methylpropyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-bromophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-cyclopentyloxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3,5-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3,5-dichlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(R)-(3,5-dichlorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-(4-tetrahydropyranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(3-methylthiopropyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2-cyclohexylethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(3(R)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-(3(S)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(3-methylbutyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2-methylcyclopropylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2(R)-tetrahydropyranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-(2(S)-tetrahydropyranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(3-methyl-3-methoxybutyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2-bicyclo[2.2.1]heptanylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-cyclobutylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-cyclopentylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(4-methyl-3(E)-pentenyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2-ethylbutyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2(R)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-(2(S)-tetrahydrofuranylmethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-cyclohexylmethoxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2,2-dimethylpropyloxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2-ethoxyethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-(2,2-diethoxyethoxy)-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-pentyloxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-isopropyloxy-9(H)-purinyl)-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2-bromophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2-bromophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-bromo-4-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(3-bromo-4-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-trifluoromethylphenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(3-trifluoromethylphenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(4-chloro-2-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(4-chloro-2-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-chloro-4-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(3-chloro-4-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2,5-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2,5-difluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2-chlorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2,4-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2,4-difluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(5-bromo-2-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(5-bromo-2-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2-chloro-4-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2-chloro-4-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(4-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(4-chlorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2,3-difluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2,3-difluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2-fluoro-5-methoxyphenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2-fluoro-5-methoxyphenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(2-trifluoromethylphenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2-trifluoromethylphenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-phenyl-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-acetyloxy-tetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-L-valinyl-tetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-isobutyryloxy-tetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-hydroxy-3(R)-methyl-4(R)-cyclopentylcarboxy-tetrahydrofuran;
2(R)-(2-Amino-6-ethoxy-9(H)-purinyl)-4(R)-(4(S)-(2,3-dichlorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(2,3-dichlorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3-chloro-5-fluorophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(3-chloro-5-fluorophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-4(R)-(4(S)-(3,5-dibromophenyl)-2-oxo-1,3,2-dioxaphosphorinan-2(R)-yloxymethyl)-1(R)-methyl-7-oxo-3,6,8-trioxa-cis-bicyclo[3.3.0]octane;
2(R)-(2-amino-6-ethoxy-9(H)-purinyl)-4(R)-(4(R)-(3,5-dibromophenyl)-2-oxo-1,3,2-dioxaphosphorinan-2(S)-yloxymethyl)-1(R)-methyl-7-oxo-3,6,8-trioxa-cis-bicyclo[3.3.0]octane;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-diacetyloxy-3(R)-methyltetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R),4(R)-dipropionyloxy-3(R)-methyltetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-methoxy-4(R)-propionyloxy-3(R)-methyltetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-methoxy-4(R)-acetyloxy-3(R)-methyltetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-fluoro-4(R)-acetyloxy-3(R)-methyltetrahydrofuran;
2(R)-(2-Amino-6-ethoxy-9(H)-purinyl)-5(R)-(4(S)-(3-chlorophenyl)-2-oxo-1,3,2-dioxaphosphorinan-2(R)-yloxymethyl)-3(R)-fluoro-4(R)-propionyloxy-3(R)-methyltetrahydrofuran;
2(R)-Uracilyl-5(R)-(4(S)-(3-chlorophenyl)-2-oxo-1,3,2-dioxaphosphorinan-2(R)-yloxymethyl)-3(R)-hydroxy-4(R)-propionyloxy-3(R)-methyltetrahydrofuran;
2(R)-Uracilyl-5(R)-(4(S)-(3-chlorophenyl)-2-oxo-1,3,2-dioxaphosphorinan-2(R)-yloxymethyl)-3(R)-hydroxy-4(R)-acetyloxy-3(R)-methyltetrahydrofuran;
2(R)-Uracilyl-5(R)-(4(S)-(3-chlorophenyl)-2-oxo-1,3,2-dioxaphosphorinan-2(R)-yloxymethyl)-3(R)-methoxy-4(R)-acetyloxy-3(R)-methyltetrahydrofuran;
2(R)-Uracilyl-5(R)-(4(S)-(3-chlorophenyl)-2-oxo-1,3,2-dioxaphosphorinan-2(R)-yloxymethyl)-3(R)-methoxy-4(R)-propionyloxy-3(R)-methyltetrahydrofuran; and
2(R)-Uracilyl-4(R)-(4(S)-(3-chlorophenyl)-2-oxo-1,3,2-dioxaphosphorinan-2(R)-yloxymethyl)-1(R)-methyl-7-oxo-3,6,8-trioxa-cis-bicyclo[3.3.0]octane.
